(19) United States
(12) Patent Application Publication
(10) Pub. No.: US 2014/0271919 A1
(43) Pub. Date: Sep. 18, 2014

(54) Topical Copper Ion Treatments and Methods of Treatment Using Topical Copper Ion Treatments in the Genital-Rectal Areas of the Body

(71) Applicants: Chun Lim Abbott, Pittsburgh, PA (US); Dominic C. Abbott, Pittsburgh, PA (US)

(72) Inventors: Chun Lim Abbott, Pittsburgh, PA (US); Dominic C. Abbott, Pittsburgh, PA (US)

(21) Appl. No.: 13/841,882

(22) Filed: Mar. 15, 2013

(51) Int. Cl. A61K 33/34

(52) U.S. Cl. CPC A61K 33/34
USPC 424/630; 604/286; 604/15; 604/60; 604/515; 128/832

(57) ABSTRACT

Copper ion treatments and methods of treatment are provided to treat various body conditions affecting the genital and/or rectal areas. The copper ion treatments contain a copper ion-containing solution which, as a result of the copper ions contacting anatomical tissue of the vagina, the rectal canal and/or the external genital or rectal areas, brings about local and systemic therapeutic effects. The copper ion treatments and methods for treating conditions affecting the genital and/or rectal areas involve the use of tampons, suppositories, lotions, creams, gels, foams and solutions, each containing a copper ion-containing solution. The tampon bodies and the copper ion lotions, creams, gels and foams may be delivered into the vagina, and the suppositories can be delivered into the vagina or into the rectal canal. The copper ion lotions, creams, gels, foams and solutions may also be topically applied to the external genital and/or rectal areas.

### FIELD OF THE INVENTION

The invention pertains generally to topical treatments containing copper ions and to methods of treating body conditions using topical treatments containing copper ions in various anatomical areas of the body. More particularly, the invention pertains to treating body conditions affecting the genital and/or rectal areas using topical treatments containing copper ions.

### BRIEF DISCUSSION OF THE RELATED ART

Many various abnormal body conditions are caused by harmful pathogens or microbes, examples of which include bacteria, fungi and viruses. Abnormal body conditions that arise in or affect the genital area in women typically affect the vagina and are commonly referred to as “vaginitis”. The term “vaginitis” encompasses infection and/or inflammation of the vagina caused by bacteria, fungi and/or viruses. Vaginitis may extend to the external female genital area, i.e. the vulva, in which case it is usually referred to as “vulvovaginitis”. In addition, bacterial, fungal and viral conditions that affect all or part of the genital area in women, i.e. vagina, vulva and/or surrounding anatomical area, may also affect all or part of the rectal (anal) area, i.e. the rectum (anal canal) and surrounding anatomical area. In men, infection and/or inflammation of bacterial, fungal and/or viral origins may affect all or part of the rectal area and also all or part of the genital area, i.e. the penis, scrotum and surrounding anatomical area.

Vaginitis that is bacterial in origin is commonly called “bacterial vaginosis”. Many different bacteria are responsible for bacterial vaginosis and some of these bacteria are the cause of sexually transmitted diseases in women and men. Examples of sexually transmitted bacterial diseases that affect the vagina and surrounding anatomical areas are gonorrhea and chlamydia, which appear in the general population on a widespread basis. It is estimated by the Centers for Disease Control and Prevention (CDC) that more than 700,000 people annually in the U.S. alone acquire new gonorrhea infections. According to the CDC, over 1.3 million chlamydia infections were recorded in the U.S. in 2010 alone. In addition, there are a large number of undiagnosed, untreated or unreported infections of gonorrhea and chlamydia because the diseases may be asymptomatic or present with only very mild symptoms. Oftentimes, gonorrhea and chlamydia occur together. Gonorrhea and chlamydia may also appear in the mouth, throat and rectum (anus) in men and women. If left untreated, gonorrhea and chlamydia can spread to the uterus and/or Fallopian tubes and may cause pelvic inflammatory disease (PID), infertility, ectopic pregnancies, chronic pelvic pain and increased risk for infection with the human immunodeficiency virus (HIV). Untreated gonorrhea may also affect the blood, joints and heart valves. The usual treatments for gonorrhea and chlamydia are appropriate antibiotics, but history has demonstrated that over time many bacterial diseases develop a resistance to antibiotics. Indeed, according to the CDC, numerous antibiotics previously used to treat gonorrhea have lost their effectiveness, and there is currently only one remaining drug, i.e. the injectable antibiotic ceftriaxone, proven effective for treating gonorrhea. There is great concern in the medical community that it is only a matter of time before gonorrhea becomes resistant to this last remaining drug. Other types of pathogens and microbes, such as the bacteria streptococcus and staphylococcus and the parasitic protozoan trichomonas, may also affect the vagina and surrounding anatomical areas resulting in abnormal biological conditions. As with gonorrhea, staphylococcus infections are especially problematic because certain strains of the bacteria have become antibiotic resistant. Infections in the vagina may spread to the uterus, resulting in PID which is often a very painful and serious condition with potentially harmful and permanent complications.

In addition to being susceptible to abnormal body conditions caused by bacteria, the vagina and surrounding anatomical areas are susceptible to various abnormal body conditions caused by viruses and fungi. Viral diseases that arise in or affect the vagina and surrounding anatomical areas include herpes (Types I and II), human papilloma virus (HPV) and HIV, all of which are sexually transmittable. Herpes, HPV and HIV can also be found in the areas of the mouth, skin and rectum (anus). Fungal diseases that arise in or affect the vagina include yeast infections, particularly candida, and thrush. Fungi are also responsible for abnormal biological conditions in other areas of the body such as the mouth (thrush), feet, skin and nails. There is no cure for herpes and HIV. Anti-viral drugs are available to alleviate herpes symptoms and suppress the herpes virus so that active infections recur less frequently and are of shorter duration, but these drugs are associated with significant side effects. Infection with HPV is usually treated with topical medications, oral medications and/or surgical removal of warts. Complications of HPV infection include increased risk for cervical, rectal and vulvar cancers. Available treatments for HIV are designed to suppress the virus and boost the immune system in hope of avoiding opportunistic infections and delaying or preventing the onset of full-blown acquired immune deficiency syndrome (AIDS). In recent years, it was hoped that a vaginal microbicide gel calcd PRO 2000 would be effective at reducing HIV infection when used shortly before sexual intercourse, but unfortunately the compound was found to be ineffective in a large scale clinical trial. Topical and oral medications are available to treat yeast and other fungal infections, but are limited in effectiveness such that fungal infections are often not eradicated and thus reoccur. The vast majority of abnormal body conditions caused by bacteria, viruses and fungi that affect the genital and/or rectal areas in women also affect the genital and/or rectal areas in men.

In addition to conditions caused by harmful pathogens or microbes, hemorrhoids are another abnormal body condition that affects the rectum (anus) in men and women and may cause rectal pain, swelling, discomfort and/or itching. Conventional treatments for hemorrhoids include topical medications and surgery. In addition to harmful microbes and pathogens, sperm are microbes that appear in the vagina after intercourse. Numerous spermicidal contraceptive compounds are available for introduction in the vagina. Typically, these must be introduced in the vagina very shortly before intercourse and are therefore oftentimes inconvenient. When intercourse takes place without contraception and there is concern for an unwanted pregnancy, drugs known as the “morning after pill” or “emergency contraceptives” are sometimes prescribed to prevent pregnancy, but these drugs are not 100% effective and may have undesirable side effects.

Abnormal body conditions of bacterial, viral and fungal origins commonly arise in dermatological areas of the body, i.e. skin and nails. The skin and soft tissue are common sites for infections caused by various bacteria including staphylococcus, enterobacter, pseudomonas, and streptocoocus. Oftentimes, infections develop on the skin at the site of a cut, scratch, abrasion, burn, splinter, boil, pimple, blister, insect bite or other wound or trauma that damages or breaks the skin or provides a point of entry for bacteria and/or other harmful organisms. Viruses such as herpes, shingles and HPV are also the cause of abnormal body conditions on the skin. In particular, herpes causes cold sores (fever blisters), shingles causes painful eruptions, and HPV causes warts on the skin. Other organisms also cause warts on the skin. The skin is susceptible to various fungal conditions, such as “athlete's foot” which commonly occurs on the feet and rashes such as ringworm. Infections of the nails, particularly fungal infections of the toenails, are also a common and tenacious problem. The skin is further susceptible to various body conditions resulting from aging, environmental factors and various external and internal causes, such conditions including sun/wind damage, dry skin, age spots, pigmentation, scarring, blisters, boils, cysts, pimples, cuts, scratches, burns, abrasions, splinters, insect bites and stings, animal bites and scratches, ulcers, loss of elasticity or collagen that manifests as wrinkles and sagging skin, acne, and many types of rashes, such as measles, chicken pox, eczema, psoriasis, impetigo and rosacea, due to various underlying external and internal causes. Various topical and oral prescription and non-prescription medications and products are available to treat the foregoing skin conditions. The skin is also a carrier for bacteria, viruses and fungi, seeing as how the skin regularly comes in contact with a plethora of pathogens and microbes. Consequently, many products such as sanitizing hand and body lotions and wipes are available commercially for the purpose of reducing germs on the skin.

The oral-respiratory-otic areas of the body, i.e. mouth, throat, nose, sinuses and ears are also common sites for abnormal body conditions due to the aforementioned pathogens and microbes. In addition, various allergies cause undesirable body conditions that impact the oral-respiratory-otic areas of the body, particularly the throat, nose and sinuses. Asthma is a chronic inflammatory disease of the airways responsible for undesirable conditions. Bacteria, viruses, fungi, allergies and/or asthma are responsible for many unwanted symptoms that appear in the oral-respiratory-otc areas of the body including sore throat, tonsilitis, colds, bronchitis, sinusitis, rhinosinusitis, wheezing, ear infections, earache, pressure in the ears, cold sores, mouth ulcers, canker sores, cough, hoarseness or laryngitis, congestion, runny nose, sneezing, sore gums, periodontal disease, tooth decay and halitosis (bad breath). A vast array of prescription and non-prescription drugs and products are commercially available to treat oral-respiratory-otic conditions.

The prescription drugs and even many of the non-prescription drugs or products used to treat the numerous body conditions described above have many drawbacks including undesirable or potentially harmful side effects, high risk of harm in the event of overdose or improper use, high cost, limited effectiveness, the need for close medical monitoring, and inconvenience. Moreover, there is presently no single compound or product to treat a wide range of body conditions affecting the genital-rectal areas that include the vagina, rectum (anus), and surrounding anatomical areas, the oral-respiratory-otic areas that include the mouth, throat, airway, nose, sinuses and ears, and the dermatological areas that include the skin and nails, much less a non-pharmaceutical topical treatment that is safe, cost-effective, easy and convenient to use, and capable of being embodied in different forms depending on the intended anatomical area or areas of use.

It has previously been established that copper possesses properties by which it is capable of killing, neutralizing and preventing the growth of human pathogens. It is known that many bacteria identified as human pathogens cannot survive on surfaces of copper metal. U.S. Pat. No. 8,135,466 B2 to Fuller at al discloses a joint prosthesis having an implant body with an external surface containing an antimicrobial metal where the antimicrobial metal may be copper. U.S. Patent Application Publications No. US 2012/0071807 A1 and No. US 2012/0089068 A1 to McClure, Jr. disclose wound dressings containing a metal-based antimicrobial agent where the metal-based antimicrobial agent may be a mixture of silver ions and copper ions. Devices having an external surface of copper metal for insertion in the vagina to treat abnormal biological conditions have been proposed by Applicants in U.S. patent application Ser. No. 12/157,823 filed Jun. 13, 2008 (abandoned), Ser. No. 13/317,230 filed Oct. 12, 2011, and Ser. No. 13/464,005 filed May 4, 2012, the entire disclosures of which are incorporated herein by reference.

Topical substances containing particles of copper or its alloys have been proposed for health support uses. A product called “MesoCopper®” sold by Purist Colloids, Inc. Is a colloidal copper solution containing nano particles of copper for use on the skin to minimize the appearance of fine lines and wrinkles. Another version of the product is sold as an ingestible mineral supplement. Copper peptides for use on the skin are also commercially available and these require peptides. i.e. small fragments of protein that have an affinity for copper to which they bind very tightly. U.S. Pat. No. 7,776,915 B2 to Morariu discloses a topical composition containing, at a minimum, a lipoic acid, a carnitine and a carnosine, where the carnosine may be chelated to zinc or copper ions. The intended use for the topical composition is to improve the appearance of aged skin. U.S. Patent Application Publication No. US2008/0195033 A1 to Eagleson et al discloses use of a metal substance to treat diseases in the body. The metal substance is primarily a colloidal suspension and delivery of the substance to the body may require the use of electricity. Prior to the present invention, it has not been recognized to provide a simple solution containing copper ions for use as a topical treatment to be applied directly to anatomical tissue to treat body conditions and/or for use in conjunction with various carriers including creams, gels, lotions, foams, pastes, other solutions, suppositories, tampons, body wipes, wound dressings, skin patches, and suture material to form topical treatments in which the carriers facilitate delivery of the copper ions to contact anatomical tissue depending on the anatomical area or areas of use on the body.

### SUMMARY OF THE INVENTION

An aspect of the invention pertains to a copper ion treatment and to methods for treating conditions affecting the genital area in women where the copper ion treatment includes a tampon body supplied with a copper ion-containing solution. Tampon bodies can be provided with different quantities of the copper ion-containing solution in order to have different potencies. The tampon body is introduced in the vagina and allowed to remain undisturbed in the vagina for a period of time, during which copper ions contact anatomical tissue of the vagina. As a result of the copper ions contacting the anatomical tissue of the vagina, local and systemic therapeutic effects are realized. The preferred number of treatments per day and the preferred number of days such treatments should be carried out can vary depending upon the quantity of copper ion-containing solution carried by the tampon bodies, the nature and severity of the underlying condition being treated, and patient history.

Another aspect of the invention pertains to a copper ion treatment and to methods of treating body conditions affecting the genital and/or rectal areas in men or women involving a suppository containing the copper ion-containing solution. The suppository can be provided in different sizes and different strengths or potencies based on the quantity of copper ion-containing solution in the suppository. The suppository is delivered into the vagina or rectum and is allowed to remain undisturbed for a period of time, during which copper ions contact anatomical tissue of the vagina or rectum and provide therapeutic effects. The preferred number of treatments per day and the preferred number of days for carrying out such treatments can vary depending upon the quantity of copper ion-containing solution in the suppositories, the nature and severity of the underlying condition being treated, and patient history.

A further aspect of the invention involves treating conditions affecting the genital area in women using copper ion treatments delivered to the vagina in the form of copper ion lotions, creams, gels or foams composed of a base material and the copper ion-containing solution. The copper ion lotions, creams, gels or foams can be provided in different strengths or potencies based on the quantity of the copper ion-containing solution contained in the copper ion lotions, creams, gels or foams. A dose of copper ion lotion, cream, gel or foam is delivered into the vagina and is allowed to remain undisturbed in the vagina for a period of time, during which copper ions contact the anatomical tissue of the vagina and provide therapeutic effects. The preferred number of treatments per day and the preferred number of days for carrying out such treatments may vary depending upon the quantity of the copper ion-containing solution contained in the copper ion lotion, cream, gel or foam, the nature and severity of the underlying condition being treated, and patient history. Conditions affecting the genital and/or rectal areas are also treated using methods involving topical application of the copper ion treatments in the form of copper ion lotion, cream, gel or foam to anatomical tissue of the external genital and/or rectal areas. The copper ion treatments and methods are used to treat body conditions affecting the genital and/or rectal areas including conditions of bacterial, viral or fungal origins. More broadly, the conditions treated using the methods described herein include gonorrhea, chlamydia, staphylococcus, streptococcus, bacterial vaginosis, herpes, HPV, genital warts, HIV, trichomonas, vaginal dryness, imbalances in vaginal pH, PID, hemorrhoids, yeast infections, candida, thrush and the risk of contracting STDs.

It is also an aspect of the invention to provide a method of female contraception using a copper ion treatment as a spermicide. A copper ion treatment composed of the copper ion-containing solution and a carrier for the solution is delivered into the vagina within a short time after sexual intercourse and is allowed to remain undisturbed in the vagina for a period of time. The copper ions in contact with the anatomical tissue of the vagina bring about a spermicidal effect. The carrier for the copper ion-containing solution may be a tampon body, a suppository, a lotion, a cream, a gel or a foam.

### BRIEF DESCRIPTION OF THE DRAWINGS

FIG. 1 is a front view of a bottle containing a copper ion treatment and having a spray pump nozzle for dispensing the copper ion treatment.

FIG. 2 is a side view of a bottle containing a copper ion treatment and having a spray pump nozzle with an elongate extension for dispensing the copper ion treatment.

FIG. 3 is a side view of a bottle containing a copper ion treatment wherein the bottle is squeezable to dispense the copper ion treatment from a dropper on the bottle.

FIG. 4 is a side view of a bottle containing a copper ion treatment and having a brush for applying the copper ion treatment to anatomical tissue.

FIG. 5 is a side view of a tube containing a copper ion treatment wherein the tube is squeezable to dispense the copper ion treatment.

FIG. 6 is a side view of an alternative bottle that is squeezable to dispense a copper ion treatment and showing the bottle in a dosed condition.

FIG. 7 is a side view of the bottle of FIG. 6 showing the bottle in an open condition.

FIG. 8 is a side view of a bottle containing a copper on treatment and having a pump nozzle for dispensing the copper ion treatment in the form of foam.

FIG. 9 is a side view of an applicator for delivering a copper ion treatment to the vagina.

FIG. 10 is a side view of the applicator of FIG. 9 showing use of the applicator in conjunction with the tube of FIG. 5.

FIG. 11 is a side view of an alternative applicator for applying a copper ion treatment onto anatomical tissue.

FIG. 12 is a side view of a tampon having a tampon body used as a carrier to deliver a copper ion treatment to the vagina.

FIG. 13 is a broken front view of a plurality of suppositories containing a copper ion treatment, the suppositories being insertable in the vagina or rectum to deliver the copper ion treatment to the vagina or rectum.

FIG. 14 is a side view showing a suppository of FIG. 13 being removed from its package.

FIG. 15 is a side view of an applicator for delivering the suppositories of FIG. 13 to the vagina or rectum.

FIG. 16 is a front view of a package containing a body wipe carrying a copper ion treatment and showing the package partially open to remove the body wipe therefrom.

FIG. 17 is a perspective view of a wound dressing supplied with a copper ion treatment.

FIG. 18 is a plan view of a skin patch carrying a copper ion treatment.

FIG. 19 is a perspective view of sutures created in anatomical tissue using suture material carrying a copper ion treatment.

FIG. 20 is a broken sectional view illustrating use of the tampon applicator of FIG. 12 to introduce the tampon body into the vagina.

FIG. 21 is a broken sectional view illustrating the applicator of FIG. 15 being used to introduce a vaginal suppository of FIG. 13 into the vagina.

FIG. 22 is a broken sectional view illustrating the applicator of FIG. 15 being used to introduce a rectal suppository of FIG. 13 into the rectal canal.

FIG. 23 is a broken sectional view showing the rectal suppository ejected from the applicator into the rectal canal.

FIG. 24 is a broken sectional view showing use of the vaginal applicator of FIGS. 9 and 10 to deliver a dose of copper ion treatment in the form of lotion, cream or gel to the vagina.

FIG. 25 is a broken sectional view showing the dose of copper ion treatment of FIG. 24 after ejection into the vagina from the vaginal applicator.

FIG. 26 is a broken top view of a dose of copper ion treatment in the form of lotion, cream, gel or foam dispensed onto the palm of a hand.

FIG. 27 is a broken side view of a dose of copper ion treatment in the form of lotion, cream, gel or foam supported on the middle and index fingers of a hand used to apply the copper ion treatment to anatomical tissue.

### DETAILED DESCRIPTION OF THE INVENTION

A solution containing copper ions, i.e. copper ion-containing solution, for use as a topical treatment containing copper ions, i.e. topical copper ion treatment, to treat body conditions is produced according to a process or method by which copper ions from copper metal are leached into an appropriate biocompatible solution. As used herein, “copper metal” means pure copper (99.5% or greater copper after processing) and copper alloys such as brasses, bronzes, copper-nickels and copper-nickel-zincs. Preferably, pure copper is used as the copper metal. Example 1 describes the steps involved in producing an amount of copper ion-containing solution equal or substantially equal to 7.44 ounces.

### Example 1

7.44 ounces of biocompatible saline solution buffered with acetic acid and sodium acetate to a pH of 5 (±0.4) is placed in a container or vessel with a tight, removable lid to minimize evaporation. The container is placed in an incubator or oven at a temperature of 37° Celsius (±1° C.). When the saline solution has reached 37° Celsius, 102 grams of pure copper metal in solid form is placed in the heated solution within the container, and the container with the tight lid thereon is placed in the incubator at 37° Celsius for 24 hours. During the 24 hour period, copper ions from the copper metal leach into the solution. At the end of the 24 hour period, the container is removed from the incubator and the copper metal is removed or separated from the solution. The amount of solution remaining after removal or separation of the copper metal therefrom constitutes the copper ion-containing solution and should be essentially 7.44 ounces with minimal evaporation. The copper ion-containing solution produced according to this process contains copper ions in an amount equal or substantially equal to 46 milligrams when analyzed for copper content by inductively coupled plasma/optical emission spectroscopy (ICP/OES). The copper ion-containing solution is stored at room temperature and is ready for use in this form as a topical copper ion treatment to be applied to anatomical tissue to treat body conditions. In addition, the copper ion-containing solution is ready for use in conjunction with various carriers including creams, gels, lotions, foams, pastes, other solutions, suppositories, tampons, body wipes, wound dressings, skin patches and suture material to form topical copper ion treatments in which the carriers facilitate delivery of the copper ion treatments to contact anatomical tissue to treat body conditions.

The solid pure copper metal in Example 1 may be in the form of one or more sheets of pure copper metal, typically in the range of 0.03 to 0.06 inch thick, of appropriate length and width to provide the 102 grams of pure copper metal. In practice, the process described in Example 1 has been carried out using as the copper metal four vaginal therapeutic devices made of pure copper in accordance with Applicants' prior patent application Ser. No. 13/464,005 previously incorporated herein by reference in its entirety. In this case, each vaginal therapeutic device used was 3.25 inches long by 0.750 inch wide with a wall thickness of 0.031 inch providing 25.5 grams of pure copper. The biocompatible saline solution used in the process described in Example 1 is commercially available from B. Braun Medical. As an alternative to the biocompatible saline, vaginal simulating fluid (VSF) buffered with acetic acid to a pH of 5 (±0.4) can be used as the biocompatible solution, but will produce less leaching of copper ions from copper metal over the 24 hour period. The VSF can be prepared in accordance with published literature, e.g. Owen, D. H., Katz, D. F., “A Vaginal Fluid Simulant”, Contraception, pages 91-95 (1999). The process described in Example 1 can be modified to eliminate the step of heating the solution prior to placement of the copper metal therein. In the latter case, the copper metal and unheated solution are placed in the container, the container with the tight lid thereon is placed in the incubator at 37° Celsius and, once the solution has reached 37° Celsius, the container with the heated solution and copper metal therein is allowed to remain in the oven for 24 hours. The copper metal can be removed or separated from the solution in various ways, such as by lifting the metal out of the solution or pouring the solution alone into another container. Of course, the quantities of biocompatible saline and solid copper mental used in Example 1 can be proportionately increased to produce a greater amount of copper ion-containing solution with each process.

The copper ion-containing solution is believed to have the greatest effectiveness for treating a wide range of body conditions when the solution contains the amount of copper ions leached into the saline from the copper metal over a 24 hour period as described in Example 1. However, it should be appreciated that the process described in Example 1 can be modified to obtain lower copper ion concentrations by adjusting the length of time that the container containing the heated saline and copper metal is allowed to remain in the incubator or oven as explained below in Examples 2, 3 and 4.

### Example 2

Follow the steps of Example 1 but allow the container containing the saline and copper metal to remain in the oven at 37° C. for one hour to obtain a copper ion-containing solution that contains an amount of copper ions equal or substantially equal to 8.8 mg.

### Example 3

Follow the steps of Example 1 but allow the container containing the saline and copper metal to remain in the oven at 37° C. for eight hours to obtain a copper ion-containing solution that contains an amount of copper ions equal or substantially equal to 22 mg.

### Example 4

Follow the steps of Example 1 but allow the container containing the saline and copper metal to remain in the oven at 37° C. for 72 hours to obtain a copper ion-containing solution that contains an amount of copper ions equal or substantially equal to 35 mg.

The copper ion-containing solution in its original form, i.e. at the end of the processes of Examples 1-4, can be applied directly to anatomical tissue in various anatomical areas of the body as a copper ion treatment to treat various body conditions. Many types of containers or bottles can be used to hold a quantity of the copper ion-containing solution and to dispense or apply the copper ion-containing solution to anatomical tissue in accordance with the intended anatomical area or areas of use. The copper ion-containing solution may also be used in conjunction with various carriers including creams, lotions, gels, foams, pastes, other solutions, tampons, suppositories, body wipes, wound dressings, skin patches and suture material to form copper ion treatments that facilitate delivery or application of the copper ion-containing solution, and therefore the copper ions, to anatomical tissue. Creams, lotions, gels, foams and pastes may be used when it is advantageous to alter the consistency of the copper ion-containing solution from its original form to obtain a thicker copper ion treatment to facilitate its delivery or application to anatomical tissue. As a result of the copper ions contacting anatomical tissue when the copper ion treatments are applied thereto, local and systemic therapeutic effects are realized including antibacterial, antimicrobial, antiseptic, antifungal, antiviral, anti-pathogenic, anti-inflammatory, spermicidal, neutralization of free radicals, promotion of healing and tissue repair, prevention of biofilm, and immune-boosting effects. In particular, these effects are realized when the copper ion treatments are used on anatomical tissue in the genital-rectal areas, the oral-respiratory-otic areas and the dermatological areas of the body since the anatomical tissue in these areas is favorable for local and systemic delivery of drugs and medicaments.

In accordance with an aspect of the present invention, the copper ion-containing solution is combined with an appropriate topical cream base to form a copper ion-containing cream, i.e. copper ion cream, in which the amount of copper ion-containing solution is preferably in the range of 5% to 30% by weight of the total weight of the copper ion cream. Examples 5, 6, 7 and 8 pertain to copper ion creams made in accordance with this aspect of the invention using the copper ion-containing solution of Example 1.

### Example 5

An appropriate amount of copper ion-containing solution is combined with a biocompatible topical cream base to form a copper ion cream in which the copper ion-containing solution constitutes 5 percent of the total weight of the copper ion cream.

### Example 6

An appropriate amount of copper ion-containing solution is combined with a biocompatible topical cream base to form a copper ion cream in which the copper ion-containing solution constitutes 10 percent of the total weight of the copper ion cream.

### Example 7

An appropriate amount of copper ion-containing solution is combined with a biocompatible topical cream base to form a copper ion cream in which the copper ion-containing solution constitutes 20 percent of the total weight of the copper ion cream.

### Example 8

An appropriate amount of copper ion-containing solution is combined with a biocompatible topical cream base to form a copper ion cream in which the copper ion-containing solution constitutes 30 percent of the total weight of the copper ion cream.

Various topical cream bases can be used as the carrier for the copper ion-containing solution in order to form the copper ion creams of Examples 5, 6, 7 and 8. One suitable topical cream base that can be used is VersaBase® cream made by Professional Compounding Centers of America (PCCA) of Houston, Tex. Another suitable topical cream base that can be used in the copper ion creams is Vanicream® made by Pharmaceutical Specialties, Inc. of Rochester, Minn. The copper ion creams are effective against the body conditions being treated when the only active ingredient in the copper ion creams directed at the underlying condition is the copper ion-containing solution. However, the copper ion creams could contain other ingredients added to the topical cream base that are not active ingredients with respect to the underlying condition being treated such as preservatives, penetrating additives, bioadhesives and stability aids. Preferably, a total weight of at least 70 grams, more preferably 80 grams, of the copper ion creams in the various strengths, i.e. 5 percent, 10 percent, 20 percent and 30 percent of copper ion-containing solution relative to the total weight of the copper ion cream, will be provided for use in containers, bottles, or tubes from which the copper ion creams can be dispensed. It should be appreciated that copper ion creams can be made using the alternative copper ion-containing solutions described above.

According to a further aspect of the present invention, a topical copper ion treatment in the form of a copper ion-containing gel, i.e. copper ion gel, is composed of the copper ion-containing solution and a suitable topical gel base as illustrated below by Examples 9, 10, 11 and 12, which utilize the copper ion-containing solution of Example 1. The amount of the copper ion-containing solution in the copper ion gel is preferably in the range of 5% to 30% by weight of the total weight of the copper ion gel.

### Example 9

An appropriate amount of copper ion-containing solution is combined with a biocompatible topical gel base to form a copper ion gel in which the copper ion-containing solution constitutes 5 percent of the total weight of the copper ion gel.

### Example 10

An appropriate amount of copper ion-containing solution is combined with a biocompatible topical gel base to form a copper ion gel in which the copper ion-containing solution constitutes 10 percent of the total weight of the copper ion gel.

### Example 11

An appropriate amount of copper ion-containing solution is combined with a biocompatible topical gel base to form a copper ion gel in which the copper ion-containing solution constitutes 20 percent of the total weight of the copper ion gel.

### Example 12

An appropriate amount of copper ion-containing solution is combined with a biocompatible topical gel base to form a copper ion gel in which the copper ion-containing solution constitutes 30 percent of the total weight of the copper ion gel.

Various topical gel bases can be used as a carrier for the copper ion-containing solution in order to form the copper ion gels. An example of a suitable topical gel base that can be used in Examples 9-12 is VersaBase® gel made by PCCA. As explained above for the copper ion creams, the copper ion gels will be effective when the only active ingredient in the copper ion gels is the copper ion-containing solution, but other ingredients that are inactive with respect to the underlying condition being treated can be added to the topical cream gels. Preferably, a total weight of at least 70 grams, more preferably 80 grams, of the copper ion gels in the various strengths, i.e. 5 percent, 10 percent, 20 percent and 30 percent of copper ion-containing solution relative to the total weight of the copper ion gel, is provided for use in containers, bottles or tubes from which the copper ion gels can be dispensed. Also, copper ion gels can be made using the alternative copper ion-containing solutions. Copper ion gels can be made having a thin, fluidic consistency, and such gels may be used as copper ion serums.

A topical copper ion treatment in the form of a copper ion-containing lotion, i.e. copper ion lotion, according to an additional aspect of the invention is composed of the copper ion-containing solution and a suitable topical lotion base as represented by Examples 13, 14, 15 and 16. Examples 13-16 employ the copper ion-containing solution of Example 1, but copper ion lotions could be made using the alternative copper ion-containing solutions. The amount of the copper ion-containing solution in the copper ion lotion is preferably in the range of 5% to 30% by weight of the total weight of the copper ion lotion.

### Example 13

An appropriate amount of copper ion-containing solution is combined with a biocompatible topical lotion base to form a copper ion lotion in which the copper ion-containing solution constitutes 5 percent of the total weight of the copper ion lotion.

### Example 14

An appropriate amount of copper ion-containing solution is combined with a biocompatible topical lotion base to form a copper ion lotion in which the copper ion-containing solution constitutes 10 percent of the total weight of the copper ion lotion.

### Example 15

An appropriate amount of copper ion-containing solution is combined with a biocompatible topical lotion base to form a copper ion lotion in which the copper ion-containing solution constitutes 20 percent of the total weight of the copper ion lotion.

### Example 16

An appropriate amount of copper ion-containing solution is combined with a biocompatible topical lotion base to form a copper ion lotion in which the copper ion-containing solution constitutes 30 percent of the total weight of the copper ion lotion.

Various topical lotion bases can be used as a carrier for the copper ion-containing solution in the copper ion lotions of Examples 13-16. One suitable topical lotion base that can be used is VersaBase® lotion made by PCCA. As explained above for the copper ion creams and gels, the copper ion lotions will be effective against the body conditions being treated when the only active ingredient in the copper ion lotions is the copper ion-containing solution, but other inactive ingredients could be added to the topical lotion base. Preferably, a total weight of at least 70 grams, more preferably 80 grams, of the copper ion lotions in the various strengths, i.e. 5 percent, 10 percent, 20 percent and 30 percent of copper ion-containing solution relative to the total weight of the copper ion lotion, will be provided for use in containers, bottles or tubes from which the copper ion lotions can be dispensed.

According to another aspect of the present invention, a topical copper ion treatment in the form of a copper ion-containing foam, i.e. copper ion foam, is composed of the copper ion-containing solution and a suitable foam base. Examples 17, 18, 19 and 20 set forth below pertain to copper ion foams or foamable solutions made in accordance with this aspect of the invention using the copper ion-containing solution of Example 1, however copper ion foams or foamable solutions can be made using the alternative copper ion-containing solutions. The amount of the copper ion-containing solution in the copper ion foam or foamable solution is preferably in the range of 5% to 30% by weight of the total weight of the copper ion foam or foamable solution.

### Example 17

An appropriate amount of copper ion-containing solution is combined with a biocompatible topical foam base to form a copper ion foam or foamable solution in which the copper ion-containing solution constitutes 5 percent of the total weight of the copper ion foam or foamable solution.

### Example 18

An appropriate amount of copper ion-containing solution is combined with a biocompatible topical foam base to form a copper ion foam or foamable solution in which the copper ion-containing solution constitutes 10 percent of the total weight of the copper ion foam or foamable solution.

### Example 19

An appropriate amount of copper ion-containing solution is combined with a biocompatible topical foam base to form a copper ion foam or foamable solution in which the copper ion-containing solution constitutes 20 percent of the total weight of the copper ion foam or foamable solution.

### Example 20

An appropriate amount of copper ion-containing solution is combined with a biocompatible topical foam base to form a copper ion foam or foamable solution in which the copper ion-containing solution constitutes 30 percent of the total weight of the copper ion foam or foamable solution.

Various topical foam bases can be used as a carrier for the copper ion-containing solution in order to form the copper ion foams or foamable solutions. Depending on the foam base used in Examples 17-20, the combination of foam base and copper ion-containing solution may be in the form of a foam. Alternatively, some foam bases that may be used will result in a foamable solution when combined with the copper ion-containing solution, and the foamable solutions will typically require an appropriate dispenser to create the actual foam. An example of a suitable topical foam base that can be used is VersaBase® foam made by PCCA. When using VersaBase® as the foam base in Examples 17-20, a foamable solution is obtained and requires a foam dispenser to create the foam. As explained above for the copper on creams, gels and lotions, the copper ion foams will be effective against the body conditions being treated with the only active ingredient therein being the copper ion-containing solution. However, other ingredients that are inactive with respect to the condition being treated can be added to the topical foam base. It is preferred that a total weight of at least 70 grams, more preferably 80 grams, of the copper ion foams or foamable solutions in the various strengths. i.e. 5 percent, 10 percent, 20 percent and 30 percent of copper ion-containing solution relative to the total weight of the copper ion foam or foamable solution, be provided in dispensers from which the copper ion foams can be dispensed.

According to a further aspect of the invention, a topical copper ion treatment in the form of a copper ion-containing paste, i.e. copper ion paste, is composed of the copper ion-containing solution and a suitable paste base. Example 21 set forth below pertains to a copper ion toothpaste made in accordance with this aspect of the invention using the copper ion-containing solution of Example 1, but copper ion pastes can also be made using the alternative copper ion-containing solutions. The amount of the copper ion-containing solution in the copper ion pastes is preferably in the range of 5% to 30% by weight of the total weight of the copper ion paste.

### Example 21

An appropriate amount of copper ion-containing solution is combined with a toothpaste base material to form a copper ion toothpaste in which the copper ion-containing solution constitutes in the range of 5 percent to 30 percent of the total weight of the copper ion toothpaste.

The toothpaste base material used in Example 21 can be a commercially available toothpaste including any of the toothpastes marketed and sold under the major brand names. A toothpaste made in accordance with Example 21 is advantageous for treating bad breath, sore gums, gum disease and tooth decay when used on a daily basis in place of a person's regular toothpaste.

According to a further aspect of the invention, the copper ion-containing solution can be combined with various base solutions to form alternative copper ion solutions. Example 22 set forth below pertains to a copper ion mouthwash made in accordance with this aspect of the invention using the topper ion-containing solution of Example 1, but copper ion solutions can also be made using the alternative copper ion-containing solutions of Examples 2-4. The amount of copper ion-containing solution in the alternative copper ion solution is preferably in the range of 5% to 30% by weight of the total weight of the copper ion solution.

### Example 22

An appropriate amount of copper ion-containing solution is combined with a mouthwash base solution to form a copper ion mouthwash in which the copper ion-containing solution constitutes in the range of 5 percent to 30 percent of the total weight of the copper ion mouthwash.

The mouthwash base solution used in Example 22 can be a commercially available mouthwash including any of the mouthwashes marketed and sold under the major brand names. A mouthwash made in accordance with Example 22 is advantageous for treating bad breath, sore gums, periodontal disease and tooth decay when used on a daily basis.

The examples described above pertaining to carriers in the nature of lotions, gels, foams and other solutions are particularly well suited for creating copper ion treatments in the nature of copper ion soaps by using as carriers lotion, gel, foam or other solution bases containing a soap component. The copper ion soaps could be designed for use as body soaps or as dish soaps.

FIG. 1 depicts a device 10 useful for dispensing the copper ion treatments, particularly the copper ion-containing solutions in their original form, e.g. the form resulting from Examples 1-4, and the copper ion lotions. The device 10 comprises a container or bottle 12 for holding the copper ion-containing solution and having a spray pump nozzle 14 with an outlet orifice 16. The spray pump nozzle 14 is resiliently biased, typically by a spring, in an upward direction away from the container 12 but is depressible in a downward direction toward the container 12 to effect the spray pump action. Each time the spray pump nozzle is manually depressed the full amount, typically using a finger of the hand holding the container, a predictable amount of copper ion-containing solution is discharged in the form of a spray or stream from the outlet orifice 16. The container 12 may include a removable protective cover 18 for being disposed over the spray pump nozzle 14 between uses. In use, the outlet orifice 16 is placed in line with anatomical tissue to be treated at a close enough distance that the tissue is within the range of the spray or stream dispensed from the outlet orifice. The spray pump nozzle 14 is then depressed the full amount using a finger, causing the predictable amount of copper ion-containing solution to be delivered or sprayed onto the anatomical tissue. The spray pump nozzle 14 can, of course, be depressed multiple times to deliver multiple sprays or streams of the copper ion-containing solution to the tissue. The device 10 is particularly useful for dispensing the copper ion-containing solution in its original form to contact anatomical tissue within the mouth and throat, anatomical tissue of the skin, and anatomical tissue of the external genital and rectal areas. The device 10 could also be adapted to dispense the copper ion lotions in a similar manner, although in such case the copper ion lotions would typically be dispensed in the form of a ribbon, mass or stream of material. In the latter case, the copper ion lotions could be dispensed directly on the tissue to be treated, or on the palm or fingers of a hand which is then used to apply the lotions on the tissue to be treated. The copper ion lotions may be best suited for use on the skin, on the external genital and rectal areas, and in the vagina.

Another device 20 useful for dispensing the copper ion treatments, particularly the copper ion-containing solution in its original form, is shown in FIG. 2. The device 20 is similar to the device 10 and comprises a container or bottle 22 having a spray pump nozzle 24 with an outlet orifice 26. The device 20, however, further includes an elongate hollow extension 28 attached to the spray pump nozzle 24. The extension 28 has a first end coupled with the outlet orifice 26 of the spray pump nozzle 24 and has an opposed second end with a wider end surface having a discharge opening 29. Preferably, a plurality of discharge openings 29 are provided along the wider end surface as shown in dotted lines in FIG. 2 to obtain a wider spray pattern as indicated by dotted lines. Each time the spray pump nozzle 24 is manually depressed the full amount, a predictable amount of copper ion treatment is released in spray form from the discharge openings 29 at the end of the extension 28. The wider end surface and plurality of discharge openings at the second end of the extension provides a wider spray pattern than the device 10. The device 20 could be designed without the spray pump nozzle, with the container 22 being squeezable to force the copper ion treatment to be discharged from the discharge opening(s) 29. The extension 28 may be selectively detachable/attachable to the spray pump nozzle 24 for ease of storage of the device 20. The device 20 may include a removable protective cover (not shown) for being placed over the nozzle 24 between uses. The device 20 is particularly useful as an atomizer for dispensing the copper ion treatments to contact anatomical tissue deeper within the mouth, throat and airway.

The device 30 depicted in FIG. 3 is also useful for dispensing the copper ion treatments, particularly the copper ion-containing solution in its original form. The device 30 comprises a squeezable container or bottle 32 for holding the copper ion treatment and having a tapered dropper or extension 34 with an outlet orifice 36 attached to a cap on the container 32. In use, the container 32 is positioned so that the outlet orifice 36, which is located at the tip of the dropper, faces anatomical tissue to be treated. The container 32 is then squeezed with the fingers and, in response to such finger pressure, individual drops of a predictable amount of copper ion treatment are released from the outlet orifice 36. Alternatively, the extension 34 can be designed to discharge the copper ion treatment in the form of a spray as shown in dotted lines in FIG. 3, which would be particularly useful as a nasal/ear spray. The tapered configuration of the dropper/extension 34 facilitates its placement in the nostril (nasal cavity) and ear (ear canal). The container 32 may include a removable protective cover 38 for being disposed over the dropper 34 between uses. The device 30 is particularly useful for dispensing the copper ion treatments to contact anatomical tissue within the nose (nostrils), ears (ear canal), skin and nails.

An additional device 40 for dispensing the copper ion treatments is shown in FIG. 4. The device 40 comprises a container or bottler 42 for holding the copper ion treatment and having a removable cap 44 with a brush 45 attached to an underside of the cap. Typically, the cap 44 will be screwed onto a neck of the container 42. When the cap 44 is disposed on the container 42, the brush 45 extends into the container and is disposed within the copper ion treatment 43. Upon removal of the cap 44 from the container 42, the cap 44 may be manipulated using the fingers and hand to contact anatomical tissue to be treated with the brush 45 in order to deposit the copper ion treatment from the brush 45 onto the anatomical tissue. The device 40 would be particularly useful for applying the copper ion treatments on the skin and nails. The brush 45 could be eliminated from the cap 44, in which case the device 40, if sized appropriately, would be advantageous for holding a copper ion solution such as a copper ion mouthwash.

The device 50 illustrated in FIG. 5 is particularly useful for dispensing the copper ion treatments formed as creams, lotions, gels and pastes. The device 50 comprises a container 52 in the form of a squeezable tube for holding the copper ion treatment and having a removable cap 54 disposed on an open end or neck 56 of the tube. Typically the cap 54 will be threaded onto an external thread 55 on the neck 56 of the tube. The cap 54 may optionally have a piercing formation 57 that may be used to puncture an optional seal covering the open neck 56 prior to the first use. Upon removal of the cap 54, the piercing formation 57 is placed against the seal, and the cap 54 is pushed in the direction of the tube 52 to puncture the seal. Once the seal is penetrated, the tube 52 can be squeezed, preferably from the bottom of the tube working upward, causing the copper ion treatment to be dispensed from the open neck 56 of the tube. The device 50 is particularly well suited for dispensing the copper ion treatments onto the fingers or palm of a hand that is then used to apply the treatments to anatomical tissue, particularly the tissue of the skin and the external genital and rectal areas. However, the copper ion treatments could be squeezed directly on the anatomical tissue to be treated. In addition, when the copper ion treatment is in a paste or other suitable form for use as a toothpaste, the device 50 is particularly well suited for dispensing the copper ion treatment onto a tooth brush in a conventional manner. As explained further below, the device 50 is particularly well suited for use with a vaginal applicator.

FIGS. 6 and 7 depict an additional device 60 useful for dispensing the copper ion treatments. The device 60 is particularly advantageous for dispensing copper ion lotions. The device 60 comprises a container or bottle 62 for holding the copper ion treatment and having a cap 64 disposed on an open end or neck of the bottle. The cap 64 could be removable or non-removable. The top surface of the cap 64 is formed by a pivotable member or disc 65 having an outlet orifice 86 along a side edge thereof. FIG. 6 depicts the cap 64 in its closed condition wherein the pivotable member 65 is in a horizontal position relative to the cap 64 and the outlet orifice 66 is disposed within the cap 64 and is not exposed. When the pivotable member 65 is depressed downwardly toward the container 62 at a location opposite the outlet orifice 66 as shown by the arrow in FIG. 7, the cap 64 will assume the open condition shown in FIG. 7 wherein the pivotable member 65 is disposed at an angle relative to the cap 64 and the outlet orifice 66 is in an exposed position located slightly above the cap 64. In use, the pivotable member 65 would be depressed using pressure applied with one or more fingers of the hand. With the cap 64 in the open condition as shown in FIG. 7, the container 62 can be squeezed manually to dispense the copper ion treatment therein from the outlet orifice 66. The cap 64 is returned to the closed position by pressing downwardly on the pivotable member 65 at a location adjacent the outlet orifice. The device 60 is advantageous for dispensing the copper ion treatments onto the palm of the hand or fingers used to apply the treatment to anatomical tissue to be treated, but the device 60 could be used to dispense the copper ion treatments directly on the anatomical tissue to be treated.

The device 70 shown in FIG. 8 is an example of a device that can be used to dispense the copper ion treatment in the form of a copper ion foam. The device 70 comprises a container 72 for holding the copper ion foam or foamable solution and having a resiliently biased foam pump dispenser 74 with an outlet orifice 76. When the foam pump dispenser 74 is depressed the full amount in a manner similar to the device 10, a predictable amount of the copper ion foam is discharged through the outlet orifice 76. If necessary, the device 70 may include a mechanism for creating foam as the copper ion treatment is discharged therefrom. The device 70 may have a removable protective cover 78 for being disposed over the foam pump dispenser 74 between uses. The device 70 could also be adapted to dispense copper ion lotions and gels.

FIG. 9 depicts a vaginal applicator 81 useful for delivering the copper ion treatments to the vagina. The vaginal applicator 81 is particularly useful in conjunction with the device 50 as depicted in FIG. 10. Also, the vaginal applicator 81 is particularly well suited for use when the copper ion treatments are in the form of either cream, lotion or gel. The vaginal applicator 81 comprises a hollow barrel 83 and a plunger 85 slidably mounted in the hollow barrel 83. The barrel 83 has an open forward end defining a discharge opening 89 and has a rearward end wall through which a stem 91 of the plunger passes. The stem 91 is attached at one end thereof to an internal flange 93 disposed within the barrel in close, sealing relation therewith. The plunger has a finger flange 95 attached to an opposite end of the stem 91 that is disposed external of the barrel 83, the flange 95 being engageable with a finger or fingers of a hand in order to selectively depress and withdraw the plunger 85 relative to the barrel 83. For use with the device 50, the forward end of the barrel 83 is provided with an internal thread 97 to threadedly engage with the external thread 55 on the neck 56 of the tube 52.

FIG. 10 illustrates the vaginal applicator 81 being filled with the copper ion treatment from the tube 52 of the device 50. As seen in FIG. 10, the cap 54 is removed from the neck 56 of the tube 52, and the forward end of the barrel 83 is threaded onto the neck 56 via threaded engagement of the threads 55 and 97. At this stage, the plunger 85 is fully withdrawn relative to the barrel 83 such that the Internal flange 93 is in abutment with the rearward end wall of the barrel 83. The tube 52 is then squeezed using pressure from the fingers in order to dispense the copper ion treatment, represented at 98, into the barrel 83 from the open neck 56 of the tube 52. When the barrel 83 is sized for a particular dosage of copper ion treatment, a sufficient amount of copper ion treatment can be dispensed from the tube 52 to entirely fill the space within the barrel 83 from the neck of the tube 56 to the Internal flange 93 which is in abutment with the rearward end wall of the barrel. Alternatively, an indicia or other marking 99 can be provided on the barrel 83 to indicate the point to which the barrel 83 should be filled with copper ion treatment 98 from the tube 52. It is preferred that filling the space within the barrel from the neck of the tube to the internal flange corresponds to a dose of 5 grams of the copper ion treatment. Once the barrel 83 has been filled with the appropriate amount of copper ion treatment 98, the barrel 83 is disengaged from the neck 56 of the tube 52 by disengaging the thread 97 from the thread 55. In order to dispense the copper ion treatment 98 from the applicator 81, the finger flange 95 of the plunger 85 is depressed toward the barrel 83 using a finger, thereby causing the internal flange 93 to push the copper ion treatment 98 through the discharge opening 89 as the plunger 85 is depressed relative to the barrel 83. When the finger flange 95 meets the rearward end wall of the barrel 83, the copper ion treatment 98 will be fully discharged from the applicator. It should be appreciated that the applicator 81 could be used in conjunction with other devices for supplying the copper ion treatments to the barrel 85. It should also be appreciated that the applicator 81 can be supplied for use pre-filled with copper ion treatment 98, in which case the forward end of the barrel would be provided with a removable cap or seal. The applicator 81 is particularly advantageous for supplying the copper ion treatments to the vagina. Accordingly, prior to depressing the plunger 85 to discharge the copper ion treatment 98 from the barrel 83, the forward end of the barrel 83 would be introduced into the vagina until the rearward end of the barrel was located near the entrance to the vagina. Then, upon depressing the plunger 85, the copper ion treatment 98 is discharged from the discharge opening 89 into the vagina.

Another type of applicator useful in applying the copper ion treatments to anatomical tissue is shown at 101 in FIG. 11. The applicator 101 is in the nature of a swab comprising a handle 103 and a body of absorbent material 105 at an end of the handle 103. The applicator 101 can be used in conjunction with a container or bottle containing a copper ion treatment, such as the device 40 of FIG. 4. Upon removal of the cap 44 from the bottle 42 of the device 40, the handle 103 of the applicator 101 can be grasped with a hand used to manipulate the applicator 101 in order to dip the body of absorbent material 105 into the copper ion treatment within the bottle 42. The body of absorbent material 105 can then be gently contacted with anatomical tissue to be treated thereby causing the copper ion treatment carried by the absorbent body 105 to be deposited on the anatomical tissue to be treated. The applicator 101 is best suited for applying copper ion treatments to localized areas of the skin, nails, ear canal, nostrils, mouth and throat. Of course, it should be appreciated that swab applicators 101 can be provided in sealed packages with the bodies of absorbent material 105 pre-supplied with copper ion treatment.

Another type of carrier that can be used to deliver copper ion treatments to the vagina is a tampon. The tampon used can be a commercially available tampon or one similar thereto. The tampon can be one having an applicator including a barrel containing the absorbent tampon body and a plunger slidable within the barrel to dispose or eject the absorbent tampon body from an open forward end of the barrel once the forward end has been introduced in the vagina an appropriate distance in a commonly known manner of tampon use. In this case, an appropriate amount of copper ion treatment can be supplied to the absorbent tampon body via the open forward end of the barrel prior to introduction of the applicator in the vagina and ejection of the absorbent tampon body from the applicator into the vagina. Another suitable tampon can be one without an applicator, i.e. a digital tampon, where the absorbent tampon body is inserted in the vagina by pushing it with the fingers. In this case, the appropriate amount of copper ion treatment is simply deposited on the absorbent tampon body prior to its insertion in the vagina. In both cases, unless the tampon is going to be inserted in the vagina immediately or soon after the absorbent tampon body has been provided with the appropriate amount of copper ion treatment, the tampon should be stored in a sealed container or package until the time of its use in order to avoid evaporation of the copper ion treatment. It should be appreciated that tampon bodies to which the copper ion treatment has been supplied can be provided in sealed containers or packages, with or without an applicator, as a ready-to-use commercial product. Alternatively, the appropriate amount of copper ion treatment may be deposited by the user on the absorbent tampon bodies of tampons sold separately or in conjunction with the copper ion treatment. Preferably, the tampon bodies are supplied with an amount of copper ion-containing solution in the range of 5 to 10 milliliters.

FIG. 12 illustrates a tampon 110 according to an aspect of the present invention including an applicator 111 having a hollow barrel 113 and a hollow plunger 115, and an absorbent tampon body 118, to which the appropriate amount of copper ion treatment has been supplied, disposed in the barrel 113 with the string 120 of the tampon body extending from a rear end of the plunger 115. The plunger 115 is slidable within and toward the barrel 113 to push the tampon body 118 and eject it from an open forward end 128 of the barrel. The forward end 128 of the barrel 113 can be tapered to facilitate introduction and advancement in the vagina and can be provided with slits that expand as the tampon body 118 passes therethrough. The tampon 110 is provided in an air-tight container or bottle 122 having a removable cap or lid 124. In order to use the tampon 110, the lid 124 is removed from the bottle 122 and the tampon 110 is removed from the bottle. The tampon 110 is inserted in the vagina in a conventional manner of using tampons. More specifically, the applicator 111 is held by grasping a finger grip 126 on the barrel 113, and the forward end 128 of the barrel is inserted in the vagina. The applicator 111 is advanced into the vagina until the fingers grasping the finger grip 126 touch the entrance to the vagina. The plunger 115 is then pushed into the barrel 113, thus causing the tampon body 118 to be ejected from the forward end 128 of the barrel into the vagina. The applicator 111 is then withdrawn from the vagina and discarded, leaving the tampon body 118 in place in the vagina. Once the tampon body 118 is in place in the vagina, the copper ion treatment carried by the tampon body contacts the anatomical tissue of the vagina and leaks into the vaginal fluid normally present in the vagina. The tampon body 118 is removed from the vagina at the appropriate time by grasping and pulling on the string 120. Examples of tampons according to an aspect of the invention are described below in Examples 23 and 24.

### Example 23

A tampon for delivering a copper ion treatment to the vagina is prepared by supplying 5 milliliters of a copper ion-containing solution to an absorbent tampon body intended to be introduced into the vagina.

### Example 24

A tampon for delivering a copper ion treatment to the vagina is prepared by supplying 10 milliliters of a copper ion-containing solution to an absorbent tampon body intended to be introduced into the vagina.

The copper ion-containing solution used in Examples 23 and 24 is the copper ion-containing solution in its original form as obtained in accordance with the method set forth in Example 1. However, it should be appreciated that tampons can be provided in which the tampon bodies are supplied with the alternative copper ion-containing solutions or other forms of the copper ion treatments.

Another type of carrier useful to deliver the copper ion treatments to the vagina and rectum is a suppository. Suppositories are commonly used in the vagina and rectum (anus) as a means for dispensing various active ingredients or medicaments. Suppositories are made in various shapes including oviform, globular, conical and bullet shapes, and in various sizes. Suppositories typically weigh in the range of 1 to 5 grams. Suppositories can be solid bodies composed of a mixture of a suitable suppository base material and the active ingredients or medicaments. Alternatively, suppositories can be made with a solid outer wall of suppository base material enclosing non-solid active ingredients or medicaments. The suppository base materials used in suppositories allow them to dissolve or melt when exposed to the moisture (body fluid) or heat (body temperature) found in the vagina or rectum (rectal or anal canal), thereby releasing the active ingredients or medicaments into the vagina or rectum. Suitable suppository base materials include oleaginous (fatty) base materials, including cocoa butter, theobroma oil and synthetic triglycerides, or water soluble or miscible base materials, including glycerinated gelatin and polyethylene glycol (PEG) polymers. It is preferred that the base materials be non-toxic, non-irritating, inert, and biocompatible. Suppositories suitable for use in an aspect of the present invention can be prepared in various ways according to conventional methods for preparing suppositories including compression molding and fusion molding. Suppositories for use as vaginal and rectal suppositories according to an aspect of the present invention are preferably made in two different sizes, i.e. a suppository weighing 3 grams and a suppository weighing 5 grams, to accommodate different sizes of vaginal and rectal anatomy. Each size suppository can be made in different strengths based on the percentage by weight of the active ingredient, i.e. the copper ion treatment, relative to the total weight of the suppository. Preferably, the amount of copper ion-containing solution in the suppository is in the range of 5% to 30% of the total weight of the suppository. The suppositories are preferably formed in plastic molds and can be stored at room temperature. The suppositories will be effective against the body condition being treated when the only active ingredient contained in the vaginal and rectal suppositories is the copper ion treatment. However, the vaginal and rectal suppositories could contain additional ingredients that are inactive with respect to the underlying condition or conditions being treated, such as preservatives, penetrating additives, bioadhesives and stability aids. The suppositories may be inserted in the vagina and rectum using the fingers, or the suppositories may be provided with applicators to facilitate insertion thereof in the vagina and rectum. Examples of vaginal and rectal suppositories according to an aspect of the invention are set forth in Examples 25-32, which utilize the copper ion-containing solution of Example 1. However, the alternative copper ion-containing solutions could be used in Examples 25-32.

### Example 25

A suppository base material is combined with an appropriate amount of copper ion-containing solution and is molded into a suppository for vaginal or rectal use having a total weight of 3 grams, wherein the copper ion-containing solution constitutes percent of the total weight of the suppository.

### Example 26

A suppository base material is combined with an appropriate amount of copper ion-containing solution and is molded into a suppository for vaginal or rectal use having a total weight of 3 grams, wherein the copper ion-containing solution constitutes percent of the total weight of the suppository.

### Example 27

A suppository base material is combined with an appropriate amount of copper ion-containing solution and is molded into a suppository for vaginal or rectal use having a total weight of 3 grams, wherein the copper ion-containing solution constitutes 20 percent of the total weight of the suppository.

### Example 28

A suppository base material is combined with an appropriate amount of copper ion-containing solution and is molded into a suppository for vaginal or rectal use having a total weight of 3 grams, wherein the copper ion-containing solution constitutes 30 percent of the total weight of the suppository.

### Example 29

A suppository base material is combined with an appropriate amount of copper ion-containing solution and is molded into a suppository for vaginal or rectal use having a total weight of 5 grams, wherein the copper ion-containing solution constitutes percent of the total weight of the suppository.

### Example 30

A suppository base material is combined with an appropriate amount of copper ion-containing solution and is molded into a suppository for vaginal or rectal use having a total weight of 5 grams, wherein the copper ion-containing solution constitutes percent of the total weight of the suppository.

### Example 31

A suppository base material is combined with an appropriate amount of copper ion-containing solution and is molded into a suppository for vaginal or rectal use having a total weight of 5 grams, wherein the copper ion-containing solution constitutes 20 percent of the total weight of the suppository.

### Example 32

A suppository base material is combined with an appropriate amount of copper ion-containing solution and is molded into a suppository for vaginal or rectal use having a total weight of 5 grams, wherein the copper ion-containing solution constitutes 30 percent of the total weight of the suppository.

FIG. 13 illustrates a strip 131 of interconnected packages or pods 132, each enclosing a vaginal or rectal suppository 130 containing a copper ion treatment. The pods 132 are separated from each other by a perforation line 133 allowing the pods 132 to be detached from each other by tearing along the perforation lines 133 as depicted in FIG. 13. Each pod 132 has front and rear walls 135 between which a suppository 130 is retained. The front and rear walls 135 are sealed to one another along their peripheral edges. As shown in FIG. 14, each pod 132 is provided with a pair of finger tabs 134 respectively attached to the front and rear walls 135, the finger tabs 134 being capable of being puled in opposite directions using the fingers to separate the opposed walls 135 and thereby release the suppository 130 contained therein.

FIG. 15 illustrates an applicator 181 suitable for use in delivering a suppository 130 to the vagina or rectum. The applicator 181 is similar to the applicator 81 but does not have an internal thread at the forward end of the barrel 183. In addition, the plunger 185 of the applicator 181 has two internal flanges 193a and 193b within the barrel 183, the flange 193a controlling the distance that the plunger can be withdrawn relative to the barrel and the flange 193b serving to eject the suppository from the barrel when the plunger is depressed the full amount. In use, a suppository 130 is manually positioned in the open forward end of the barrel 183 as illustrated in FIG. 15. The open forward end of the barrel 183 is preferably sized to retain the suppository 130 in position without being overly snug or tight. The plunger 185 is withdrawn the full amount relative to the barrel 183, which coincides with abutment of internal flange 193a with the rearward end wall of the barrel 183. The forward end of the barrel 183 holding the suppository is then introduced in the vagina or rectal (anal) canal, and the applicator 181 is gently pushed into the vagina or rectal canal until the fingers holding the rearward end of the barrel 183 are adjacent or touch the entrance to the vagina or rectal canal. The finger flange 195 is then depressed to push the plunger 185 toward and into the barrel 183 as shown by the arrow in FIG. 15, thus causing the flange 193b to engage the suppository 130 and eject it from the forward end of the barrel into the vagina or rectal canal. The applicator 181 is then removed from the vagina or rectal canal, leaving the suppository in the vagina or rectum. The suppository will melt or dissolve in the vagina or rectal canal such that the copper ion treatment is released to contact anatomical tissue of the vagina or rectal canal and to mingle with body fluid present in the vagina or rectal canal.

Another type of carrier that can be used to deliver the copper ion treatments to anatomical tissue is a body wipe. FIG. 16 illustrates a body wipe 200 contained in a sealed package 202 having front and rear walls 203. The body wipe 200 comprises a thin sheet of material disposed in a folded condition when retained between the front and rear walls 203, which are sealed along their peripheral edges. The body wipe 200 enclosed between the front and rear walls 203 contains a wet or moist copper ion treatment. The front and rear walls 203 may be grasped by the fingers at corresponding corners thereof and pulled in opposite directions similar to the pods 132 in order to separate the front and rear walls 203 and thereby allow the body wipe 200 to be removed from the package 202. FIG. 16 shows the package 202 in a partially open condition in which corresponding corner sections of the front and rear walls 203 have been peeled away from one another thereby providing access to the body wipe 200. Upon removal from the package 202, the body wipe 200 can be unfolded to its full size, which is substantially larger than its size in the folded condition, and can be used to wipe anatomical tissue to be treated causing the copper ion treatment to be transferred to the anatomical tissue. The body wipe 200 is advantageous for applying the copper ion treatments to the skin and the external genital and rectal areas.

Another type of carrier for the copper ion treatments is a wound dressing, such as a band aid, gauze pad or similar device. Such carriers can be selected from products that are commercially available for removable application to the skin to temporarily cover and protect an affected area of the skin. FIG. 17 depicts a carrier in the nature of a wound dressing 300 having a surface 301 for being placed in contact with the skin. The surface 301 includes a protective surface 302 for being positioned over a wound, and an adhesive border 303 surrounding the surface 302 in use, a copper ion treatment, such as the copper ion-containing solution in original form, can be liberally sprayed onto the surface 302 of the carrier that is applied adjacent or in contact with the skin. Then, when the surface 302 of the carrier is applied adjacent or in contact with the skin and the carrier is left in place on the skin for a period of time, the copper ions contact or are transferred to the skin and provide the therapeutic effects described above. Of course, it would be possible to provide carriers of this type in sealed packages in which the carriers are pre-supplied or pre-treated with the copper ion treatment similar to the body wipe 200.

A further type of carrier for the copper ion treatments is a skin patch, such as a dermal patch or a transdermal patch, represented at 400 in FIG. 18. The skin patch 400 has a drug delivery surface 401 containing the copper ion treatment surrounded by an adhesive border 402. The patch is applied to the skin and is left in place for a period of time with the drug delivery surface in contact with the skin, causing the copper ions to diffuse through the skin where they can act locally or penetrate the capillaries for broader systemic effects. Examples of suitable transdermal patches are the transdermal and microneedle 3M Drug Delivery Systems manufactured by 3M corporation.

An additional type of carrier for the copper ion treatments is suture material, represented at 400 in FIG. 19, used by medical professionals to close or suture external or internal incisions or wounds, i.e. “stitches.” Prior to using the suture material 500, which can be conventional suture material, the suture material 500 can be soaked in the copper ion-containing solution for a period of time in order to cover or saturate the suture material with the solution. Then, when the suture material 500 is used to create sutures or stitches in anatomical tissue T as seen in FIG. 19, the copper ions in the solution contact the anatomical tissue and provide the therapeutic effects previously described.

The copper ion-containing solution and the other forms of copper ion treatments described herein can be used on anatomical tissue in various areas of the body including the genital-rectal areas (vagina, vulva, penis, scrotum, rectum (anus) and surrounding anatomical areas), the oral-respiratory-otic areas (mouth, throat, airway, nostrils and ears) and the dermatological areas (skin and nails) of the body. The treatment effects provided by the copper ion treatments encompass treatment of active or existing disease and other undesirable body conditions as well as the prevention of such diseases and conditions. The copper ion treatments are especially beneficial for their ability to kill or neutralize harmful or undesired pathogens and microbes including bacteria, viruses and fungi. Although the copper ion treatments are applied topically to anatomical tissue and have a localized effect on diseases and undesirable body conditions affecting the anatomical tissue, the copper ion treatments also have a broader systemic effect on diseases and undesirable body conditions. The effects realized with the copper ion treatments include antibacterial, antimicrobial, antiseptic, antifungal, antiviral, anti-pathogenic, anti-inflammatory, spermicidal, neutralization of free radicals, promotion of healing and tissue repair, prevention of biofilm, and immune-boosting effects. The diseases or conditions affecting the genital-rectal areas that are treatable with the copper ion treatments include vaginitis, bacterial vaginosis, hemorrhoids, vaginal dryness, imbalances in vaginal pH, bacterial infections caused by gonorrhea, chlamydia, streptococcus and staphylococcus, protozoan infections caused by trichomonas, pelvis inflammatory disease, viral infections caused by herpes (I and II), HPV and HIV, fungal infections caused by yeast, candida, thrush and other fungi, exposure to sexually transmitted diseases, and the risk of undesired pregnancy (contraception). The diseases or conditions affecting the oral-respiratory-otic areas that are treatable with the copper ion treatments include bacterial infections caused by gonorrhea, chlamydia, streptococcus and staphylococcus, protozoan infections caused by trichomonas, viral infections caused by herpes (I and II), HPV and HIV, canker sores, mouth sores, mouth ulcers, colds, sinusitis, rhinosinusitis, sore throat, nasal discharge, congestion, runny nose, bronchitis, allergies, asthma, tonsillitis, wheezing, sneezing, ear infections, earache, pressure in the ears, cough, hoarseness, laryngitis, sore gums, periodontal disease, bad breath and tooth decay. The diseases or conditions affecting the dermatological areas that are treatable with the copper ion treatments include bacterial infections caused by staphylococcus, streptococcus, enterobacter, E. coli, and pseudomonas, viral infections caused by shingles, herpes (I and II) and HPV, fungal infections such as athlete's foot, ringworm and toenail fungus, impetigo, rosacea, psoriasis, eczema, warts, sun/wind damage, dry skin, age spots, pigmentation, scarring, blisters, boils, cysts, pimples, cuts, scratches, burns, abrasions, splinters, insect bites and stings, animal bites and scratches, ulcers, loss of elasticity or collagen, wrinkles, sagging skin, acne, measles, chicken pox, and the presence of pathogens and microbes on the skin that is an inevitable consequence of daily life. Based on the result of laboratory testing, it is expected that the copper ion treatments will kill bacteria causing bacterial vaginosis, gonorrhea and chlamydia, and the viruses responsible for herpes (I and II) and HIV at a kill rate of 99.99 percent in 6 hours. Accordingly, the copper ion treatments are sufficiently effective to “cure” the diseases and conditions described herein and to prevent the occurrence or development of such diseases and conditions. Similarly, copper has been demonstrated as having the capability to kill or render inactive staphylococcus, streptococcus, enterobacter, trichomonas, E. coli and pseudomonas. The copper ion treatments are highly effective at treating the various abnormal or undesired body conditions while being safe and non-toxic. In particular, copper toxicity is so rare that the World Health Organization (WHO) has determined that there is no need for setting an upper threshold for the ingestion of copper. The copper ion treatments can thus be safely used without concern for overdosing or improper use. Moreover, it is believed that, to date, no bacteria or other harmful microorganisms have been found to be capable of developing a resistance to copper, in contrast to the many bacteria and organisms that have developed or are in the process of developing resistance to conventional antibiotics. The multi-target effects of copper makes bacterial resistance extremely unlikely as copper kills bacteria very quickly and leaves almost no survivors. Consequently, there is neither the time for bacteria to “learn” how to resist the killing effect of copper or the possibility to pass on any knowledge to a significant population of survivors. The copper ion treatments provide a degree of efficacy and safety for treating a wide array of diseases and body conditions that far surpasses conventional pharmaceutical and non-pharmaceutical products and drugs available for treating the same conditions.

According to an aspect of the invention, vaginitis is treated using the tampon 110 having the tampon body 118 that contains or carries a copper ion treatment. A method of treating vaginitis involves introducing the tampon body 118 into the vagina as previously described above and as illustrated in FIG. 20. FIG. 20 shows the barrel 113 of the applicator 111 introduced in the vagina V and the plunger 115 being depressed to eject the tampon body 118 into the vagina as previously explained above. Once the tampon body 118 is completely ejected from the barrel, the applicator 111 is withdrawn, leaving the tampon body 118 in place in the vagina. A method of treating vaginitis using the tampon 110 having a tampon body 118 prepared in accordance with Example 23 is described below in Example 33.

### Example 33

At the beginning of a 24 hour period as soon as possible following diagnosis or the onset of symptoms, insert into the vagina V a first tampon body 118 that has been supplied with 5 milliliters (ml) of the copper ion-containing solution. Allow the tampon body 118 to remain undisturbed in the vagina for 6 to 8 hours. Remove the tampon body 118 from the vagina after 6 to 8 hours and discard it in a wastebin or by flushing it down the toilet. Insert into the vagina V a second tampon body 118 that has been supplied with 5 ml of the copper ion-containing solution and allow it to remain in the vagina for 6 to 8 hours within the same 24 hour period. Remove the second tampon body 118 from the vagina after 6 to 8 hours and discard it in a wastebin or by flushing it down the toilet. Repeat the preceding steps for the next subsequent 24 hour period and each subsequent 24 hour period thereafter until first and second tampon bodies have been allowed to remain in the vagina for 6 to 8 hours within each 24 hour period or day for seven consecutive days or seven consecutive 24 hour periods.

A method of treating vaginitis using the tampon 110 having a tampon body 118 prepared in accordance with Example 24 is described below in Example 34.

### Example 34

At the beginning of a 24 hour period as soon as possible following diagnosis or the onset of symptoms, insert into the vagina V a tampon body 118 that has been supplied with 10 ml of the copper ion-containing solution. Allow the tampon body 118 to remain in the vagina undisturbed for 6 to 8 hours. Remove the tampon body 118 from the vagina V after 6 to 8 hours and discard it in a wastebin or by flushing it down the toilet. Repeat the preceding steps for the next subsequent 24 hour period and each subsequent 24 period thereafter until a tampon body has been allowed to remain in the vagina for 6 to 8 hours within each 24 hour period or day for seven consecutive days or seven consecutive 24 hour periods.

In the method described in Example 33, it is preferred that one of the tampon bodies in each 24 hour period be inserted in the vagina at bedtime and be removed from the vagina in the morning. The second tampon can be inserted in the morning, a short while after removing the first tampon. In the method described in Example 34, it is preferred that each tampon body inserted in the vagina during a 24 hour period be inserted in the vagina at bedtime and be removed from the vagina in the morning. The choice between the methods of Examples 33 and 34 can be made with the advice of a medical professional based on factors such as the underlying condition or conditions causing the vaginitis, severity of the condition(s) and patient history. A sanitary pad or panty liner can be used in the underwear in a conventional manner to absorb any leakage from the vagina. The method described in Example 33 can also be used as a method of female contraception, particularly emergency contraception. In this case, the method should be initiated as soon as possible, i.e. within 4 to 6 hours and preferably sooner, after the act of sexual intercourse that raises a concern for pregnancy.

As a result of the copper ions in the copper ion-containing solution contacting the anatomical tissue of the vagina V and mixing with the vaginal body fluids, local (vaginal) and systemic effects are realized including antibacterial, antimicrobial, antiseptic, antifungal, antiviral, anti-pathogenic, anti-inflammatory, spermicidal, neutralization of free radicals, promotion of healing and tissue repair, prevention of biofilm, and immune-boosting effects. The local and systemic effects are believed to be due to the fact that its large surface area, rich blood supply, permeability, and potential for direct local transfer from the vagina to the uterus make the vagina a favorable site for local and systemic delivery of drugs, albeit it has been previously under-explored and under-utilized for this purpose. In the vagina, it is believed that the copper ions act across the vaginal membrane by the transcellular route, intracellular route or vesicular and receptor-mediated transport mechanisms to bring about the aforementioned beneficial effects. In addition, vaginal delivery avoids the filtering effects provided by the kidneys and liver on orally delivered medications.

According to another aspect of the invention, vaginitis is treated using a vaginal suppository 130 and applicator 181 as previously described above and as illustrated in FIG. 21. FIG. 21 shows the barrel 183 of the applicator 181 introduced in the vagina V and the plunger 185 of the applicator being depressed to eject the suppository 130 from the barrel 183 into the vagina V as previously explained above. Once the suppository 130 is completely ejected from the barrel, the applicator 181 is withdrawn, leaving the suppository 130 in place in the vagina V. Methods of treating vaginitis using suppositories 130 prepared in accordance with Examples 25-32 are described below in Examples 35-38.

### Example 35

At the beginning of a 24 hour period as soon as possible following diagnosis or the onset of symptoms, insert into the vagina V a first vaginal suppository 130 of either 3 gram or 5 gram size, depending on the size of the vagina V, and having 5 percent by weight of the copper ion-containing solution (Examples 25 and 29). Allow the first vaginal suppository to remain undisturbed in the vagina for 6 to 8 hours. After the first suppository has remained undisturbed in the vagina for at least 6 to 8 hours, insert into the vagina V a second vaginal suppository 130 of the same size and strength as the first vaginal suppository and allow the second suppository to remain undisturbed in the vagina for at least 6 to 8 hours within the same 24 hour period such that two vaginal suppositories 130 are inserted in the vagina V within a single 24 hour period or day with at least 6 to 8 hours between insertions. Repeat the preceding steps for the next subsequent 24 hour period and each subsequent 24 hour period thereafter until first and second vaginal suppositories have been allowed to remain undisturbed in the vagina for 6 to 8 hours within each 24 hour period or day for 10 consecutive days or 10 consecutive 24 hour periods.

### Example 36

Follow the steps described in Example 35 but using vaginal suppositories 130 of either 3 gram or 5 gram size and having 10 percent by weight of the copper ion-containing solution (Examples 26 and 30).

### Example 37

At the beginning of a 24 hour period as soon as possible following diagnosis or the onset of symptoms, insert into the vagina V a first vaginal suppository 130 of either 3 gram or 5 gram size, depending on the size of the vagina V, and having 20 percent by weight of the copper ion-containing solution (Examples 27 and 31). Allow the first vaginal suppository to remain undisturbed in the vagina for 6 to 8 hours. After the first suppository has remained undisturbed in the vagina for at least 6 to 8 hours, insert into the vagina a second vaginal suppository 130 of the same size and strength as the first suppository and allow the second suppository to remain undisturbed in the vagina for at least 6 to 8 hours within the same 24 hour period such that two vaginal suppositories 130 are inserted in the vagina V within a single 24 hour period or day with at least 6 to 8 hours between insertions. Repeat the preceding steps for the next subsequent 24 hour period and each subsequent 24 hour period thereafter until first and second suppositories have been allowed to remain undisturbed in the vagina for 6 to 8 hours within each 24 hour period or day for seven consecutive days or seven consecutive 24 hour periods.

### Example 38

At the beginning of a 24 hour period as soon as possible following diagnosis or the onset of symptoms, insert into the vagina V a vaginal suppository 130 of either 3 gram or 5 gram size, depending on the size of the vagina, and having 30 percent by weight of the copper ion-containing solution (Examples 28 and 32). Allow the suppository to remain undisturbed in the vagina for at least 8 to 8 hours. Repeat the preceding step for the next 24 hour period and each subsequent 24 hour period thereafter until a vaginal suppository has been allowed to remain undisturbed in the vagina for 6 to 8 hours within each 24 hour period or day for seven consecutive days or seven consecutive 24 hour periods.

When treating vaginitis according to the methods described in Examples 35, 36 and 37, it is preferred that one of the suppositories for each 24 hour period be inserted in the vagina at bedtime and that the other suppository for the 24 hour period be inserted in the vagina in the morning. When treating vaginitis according to the method described in Example 38, it is preferred that the suppository for each 24 hour period be inserted in the vagina at bedtime. Because the vaginal suppositories 130 dissolve or melt once inserted into the vagina V, thereby releasing the copper ions to contact the anatomical tissue of the vagina and mix with vaginal fluid, there is no need to subsequently remove anything from the vagina. As with the tampons described above, a sanitary pad or panty liner can be used in the underwear in a conventional manner to absorb any leakage from the vagina when suppositories are used. As explained above for Example 33, the method of Example 37 can also be used as a method of female contraception when initiated as soon as possible after the act of sexual intercourse giving rise to a risk of pregnancy.

The methods described in Examples 35-38 can be modified such that the suppositories 130 are used as a copper ion treatment delivered rectally instead of vaginally as illustrated in FIGS. 22 and 23. As shown in FIGS. 22 and 23, methods involving rectal delivery of the suppositories 130 involve introducing the barrel 183 of the applicator 181 into the rectal (anal) canal R with a rectal suppository 130 disposed in the open forward end of the barrel as seen in FIG. 22. Once the barrel 183 has been introduced into the rectal canal R the appropriate distance, the plunger 115 is depressed to eject the rectal suppository 130 from the barrel into the rectal canal R as depicted in FIG. 23 and as previously explained in detail above. Once the rectal suppository 130 is completely ejected into the rectal canal R, the applicator 181 is withdrawn, leaving the rectal suppository 130 in the rectal canal as shown in FIG. 23. Methods of treatment involving rectal delivery of suppositories 130 are used primarily to treat bacterial, viral and/or fungal conditions affecting the genital area in men and bacterial, viral and/or fungal conditions affecting the rectal area in men and women. Although the vagina is the preferred site for delivery of the copper ion treatments when treating vaginitis, it should be appreciated that vaginitis can also be treated by delivering the copper ion treatments to the rectal canal. In particular, the local and systemic effects realized when the copper ion treatments are delivered vaginally are also realized when the copper ion treatments are delivered rectally, as the anatomical tissue and environment of the rectal (anal) canal are similarly favorable to local and systemic delivery of drugs and therapeutic substances. Methods of treating bacterial, viral and/or fungal conditions affecting the genital and/or rectal areas in men and women using the rectal suppositories 130 are explained below in Examples 39-42.

### Example 39

At the beginning of a 24 hour period as soon as possible following diagnosis or the onset of symptoms, insert into the rectal (anal) canal R a first rectal suppository 130 of either 3 gram or 5 gram size, depending on the size of the rectal canal R, and having 5 percent by weight of copper ion-containing solution (Examples 25 and 29). Allow the first rectal suppository to remain undisturbed in the rectal canal for 6 to 8 hours. After the first suppository has remained undisturbed in the rectal canal for at least 6 to 8 hours, insert into the rectal canal R a second rectal suppository 130 of the same size and strength as the first rectal suppository and allow the second suppository to remain undisturbed in the rectal canal for at least 6 to 8 hours within the same 24 hour period such that two rectal suppositories 130 are inserted in the rectal canal R within a single 24 hour period or day with at least 6 to 8 hours between insertions. Repeat the preceding steps for the next subsequent 24 hour period and each subsequent 24 hour period thereafter until first and second rectal suppositories have been allowed to remain undisturbed in the rectal canal for 6 to 8 hours within each 24 hour period or day for 10 consecutive days or 10 consecutive 24 hour periods.

### Example 40

Follow the steps described in Example 39 but using rectal suppositories 130 of either 3 gram or 5 gram size and having 10 percent by weight of the copper ion-containing solution (Examples 26 and 30).

### Example 41

At the beginning of a 24 hour period as soon as possible following diagnosis or the onset of symptoms, insert into the rectal canal R a first rectal suppository 130 of either 3 gram or 5 gram size, depending on the size of the rectal canal R, and having 20 percent by weight of the copper ion-containing solution (Examples 27 and 31). Allow the first rectal suppository to remain undisturbed in the rectal canal for 6 to 8 hours. After the first suppository has remained undisturbed in the rectal canal for at least 6 to 8 hours, insert into the rectal canal R a second rectal suppository 130 of the same size and strength as the first suppository and allow the second suppository to remain undisturbed in the rectal canal for at least 6 to 8 hours within the same 24 hour period such that two rectal suppositories 130 are inserted in the rectal canal within a single 24 hour period or day with at least 6 to 8 hours between insertions. Repeat the preceding steps for the next subsequent 24 hour period and each subsequent 24 hour period thereafter until first and second rectal suppositories have been allowed to remain undisturbed in the rectal canal for 6 to 8 hours within each 24 hour period or day for seven consecutive days or seven consecutive 24 hour periods.

### Example 42

At the beginning of a 24 hour period as soon as possible following diagnosis or the onset of symptoms, insert into the rectal canal R a rectal suppository 130 of either 3 gram or 5 gram size, depending on the size of the rectal canal, and having 30 percent by weight of the copper ion-containing solution (Examples 28 and 32). Allow the suppository to remain undisturbed in the rectal canal for at least 6 to 8 hours. Repeat the preceding step for the next 24 hour period and each subsequent 24 hour period thereafter until a rectal suppository has been allowed to remain undisturbed in the rectal canal for 6 to 8 hours within each 24 hour period or day for seven consecutive days or seven consecutive 24 hour periods.

In the methods described in Examples 39, 40 and 41, it is preferred that one of the suppositories for each 24 hour period be inserted in the rectal canal at bedtime and that the other suppository for the 24 hour period be inserted in the rectal canal in the morning. In the method described in Example 42, it is preferred that the suppository for each 24 hour period be inserted in the rectal canal at bedtime. Because the rectal suppositories 130 dissolve or melt once inserted into the rectal canal, thereby releasing the copper ions to contact the anatomical tissue of the rectum (anus) and mix with the fluid/mucous present in the rectal canal, there is no need to subsequently remove anything from the rectal canal. As pointed out above, a sanitary pad or panty liner can be used in the underwear in a conventional manner to absorb any leakage from the rectum when suppositories are used.

It is a further aspect of the present invention to treat vaginitis using the copper ion lotions, creams, gels and foams delivered to the vagina using a vaginal applicator. FIG. 24 shows the barrel 83 of the vaginal applicator 81, which has been filled with a 5 gram dose of copper ion treatment 98 in the form of copper ion lotion, cream or gel as previously described above, introduced in the vagina V and showing the plunger 85 being depressed to eject the dose of copper ion treatment 98 from the barrel into the vagina V. Once the plunger 85 is depressed the full amount, the applicator 81 is withdrawn, leaving the dose of copper ion treatment 98 in the vagina V as illustrated in FIG. 25. Methods of treating vaginitis using a copper ion treatment in the form of a copper ion lotion, cream, gel or foam are described below in Examples 43, 44, 45 and 46.

### Example 43

At the beginning of a 24 hour period as soon as possible following diagnosis or the onset of symptoms, deliver into the vagina V a first dose of a copper ion treatment in the form of copper ion lotion, cream, gel or foam equal or substantially equal to 5 grams and having 5 percent by weight of the copper ion-containing solution (Examples 5, 9, 13 and 17). Allow the first dose of copper ion treatment to remain in the vagina undisturbed for 6 to 8 hours. After the first dose of copper ion treatment has remained undisturbed in the vagina for at least 6 to 8 hours, deliver into the vagina V a second dose of the copper ion treatment equal or substantially equal to 5 grams and allow the second dose to remain undisturbed in the vagina for at least 6 to 8 hours within the same 24 hour period such that two doses of copper ion treatment are delivered to the vagina V within a single 24 hour period or day with at least 6 to 8 hours between doses. Repeat the preceding steps for the next subsequent 24 hour period and each subsequent 24 hour period thereafter until first and second doses of the copper ion treatment have been allowed to remain undisturbed in the vagina for 6 to 8 hours within each 24 hour period or day for 10 consecutive days or 10 consecutive 24 hour periods.

### Example 44

Follow the steps described in Example 43 but using a copper ion treatment in the form of copper ion lotion, cream, gel or foam having 10 percent by weight of the copper ion-containing solution (Examples 6, 10, 14 and 18).

### Example 45

At the beginning of a 24 period as soon as possible following diagnosis or the onset of symptoms, deliver into the vagina V a first dose of copper ion treatment in the form of copper ion lotion, cream, gel or foam equal or substantially equal to 5 grams and having 20 percent by weight of the copper ion-containing solution (Examples 7, 11, and 19). Allow the first dose of copper ion treatment to remain in the vagina undisturbed for 6 to 8 hours. After the first dose of copper ion treatment has remained undisturbed in the vagina for at least 6 to 8 hours, deliver into the vagina a second dose of the copper ion treatment equal or substantially equal to 5 grams and allow the second dose to remain undisturbed in the vagina for at least 6 to 8 hours within the same 24 hour period such that two doses of copper ion treatment are delivered to the vagina V within a single 24 hour period or day with at least 6 to 8 hours between doses. Repeat the preceding steps for the next subsequent 24 hour period and each subsequent 24 hour period thereafter until first and second doses of the copper ion treatment have been allowed to remain undisturbed in the vagina for 6 to 8 hours within each 24 hour period or day for seven consecutive days or seven consecutive 24 hour periods.

### Example 46

At the beginning of a 24 hour period as soon as possible following diagnosis or the onset of symptoms, deliver into the vagina V a dose of copper ion treatment in the form of copper ion lotion, cream, gel or foam equal or substantially equal to 5 grams and having 30 percent by weight of the copper ion-containing solution (Examples 8, 12, 16 and 20). Allow the dose of copper ion treatment to remain undisturbed in the vagina for 6 to 8 hours. Repeat the preceding steps for the next subsequent 24 hour period and each subsequent 24 hour period thereafter until a single dose of copper ion treatment has been allowed to remain undisturbed in the vagina for 6 to 8 hours within each 24 hour period or day for seven consecutive days or seven consecutive 24 hour periods.

When treating vaginitis according to the methods described in Examples 43, 44 and 45, it is preferred that one of the doses for each 24 hour period be delivered to the vagina at bedtime and that the other dose for the 24 hour period be delivered to the vagina in the morning. When treating vaginitis according to the method described in Example 46, it is preferred that the dose for each 24 hour period be delivered to the vagina at bedtime. As with the tampons and suppositories described above, a sanitary pad or panty liner can be used in the underwear in a conventional manner to absorb any leakage of copper ion lotion, cream, gel or foam from the vagina. As described above for Examples 33 and 37, the method of Example 45 can be used as a method of female contraception when initiated as soon as possible after an act of sexual intercourse giving rise to a risk of pregnancy.

Another aspect of the present invention involves treating conditions affecting the external genital and/or rectal areas in men and women using copper ion treatments directly topically applied to the external genital and/or rectal areas. Many conditions affecting the external genital and/or rectal areas can be treated including conditions of bacterial, viral and/or fungal origins. Copper ion treatments in the form of copper ion solutions, lotions, creams, gels and foams may be best suited for external application. When using the copper ion-containing solution in its original form or in combination with another solution, the solution can simply be sprayed onto the external genital and/or rectal area and allowed to air dry. The device 10 of FIG. 1 would be useful for this purpose. When using a copper ion treatment in the nature of a copper ion soap, the soap can be applied to the external genital and/or rectal areas using the fingers to apply the soap and gently rub it into the anatomical tissue, and the excess soap can be rinsed away using clean water. Typically, a copper ion treatment in the form of copper ion lotion, cream, gel or foam will be topically applied to external anatomical tissue of the genital and/or rectal area using the fingers of a hand as represented in FIGS. 26 and 27 FIG. 26 shows a dose of copper ion treatment 98 in the form of copper ion lotion, cream, gel or foam deposited on the palm P of a hand H. The dose is a dollop of copper ion lotion, cream, gel or foam in the approximate size of a nickel or quarter. The dose can be delivered or deposited onto the palm P of the hand H from a device such as the devices 10, 50, 60 and 70 previously described above. The fingers F of the opposite hand may be used to “scoop” the dollop of copper ion treatment from the palm P, as seen in FIG. 27 which shows the dose of copper ion treatment 98 now deposited on the index and middle fingers F of the opposite hand H. Alternatively, the copper ion treatment can be dispensed or deposited directly onto the fingers F of the hand. Using the fingers F, the copper ion treatment 98 can be applied to anatomical tissue of the external genital and/or rectal areas and gently rubbed into the tissue. Methods of treating conditions affecting the external genital and/or rectal area using copper ion treatments applied directly to the external tissue of the genital and/or rectal area are explained below in Example 47.

### Example 47

Clean and dry the external genital and/or rectal area to which the copper ion treatment is to be applied, particularly areas having visible signs of inflammation such as infection, irritation or disease. Deposit a nickel or quarter size dose or dollop of copper ion treatment in the form of a copper ion lotion, cream, gel or foam onto the fingers of a hand. Use the fingers to apply the copper ion treatment to the external genital and/or rectal area. Gently pat or rub the copper ion treatment into the anatomical tissue. Repeat once or twice daily as needed to alleviate inflammation and/or prevent inflammatory conditions.

The method of Example 47 can be modified so that the copper ion treatment is sprayed directly on the tissue of the external genital and/or rectal area. When the copper ion treatment used in Example 47 is a copper ion soap, the initial step of cleaning and drying the external area can be omitted, and the step of rinsing away the excess soap following its application to the tissue can be added. In women, the method described in Example 47 may involve applying the copper ion treatments to any part of the external genital area including the vaginal opening, the vulva and surrounding areas, and any part of the rectal area including the rectal opening and surrounding rectal area. In men, the method described in Example 47 may involve applying the copper ion treatments to any part of the genital area including the penis, the scrotum and surrounding areas, and any part of the rectal area including the rectal opening and surrounding rectal area. Because the copper ion treatments are non-toxic and highly safe, the copper ion solutions, lotions, creams, gels and foams can be liberally applied and can be applied frequently to treat undesired conditions as well as to prevent such conditions from developing. When treating active conditions, treatment should be initiated as soon as possible following diagnosis or onset of symptoms.

Another way in which the copper ion treatment can be topically applied to external anatomical tissue of the genital and/or rectal areas involves the use of the body wipe 200 described above and illustrated in FIG. 16. A method of treatment involving use of the body wipe 200 is explained below in Example 48.

### Example 48

The body wipe 200 is removed from the package 202 and unfolded. The body wipe 200 is then used to thoroughly wipe the external genital area and/or rectal area, thereby depositing the copper ions on the anatomical tissue. The anatomical area is allowed to air dry. The method may be repeated once or twice daily or more often, as needed, to alleviate symptoms and/or to maintain good health in the genital and/or rectal areas. The method may be carried out on a regular basis to eliminate or reduce the presence of harmful pathogens and microbes in the genital and/or rectal areas and thereby prevent the development of undesired body conditions.

It is also an aspect of the present invention to use the copper ion treatments as a treatment for odor arising in or from the genital and/or rectal areas. Any of the Examples described above in which the copper ion treatments are delivered vaginally, rectally or topically to the external genital and/or rectal areas will be effective at eliminating or reducing offensive odor arising in or from the genital and/or rectal areas. In addition, the copper ion-containing solution can be sprayed onto portions of various products, such as panty liners, sanitary pads, tampons, incontinence underproducts and/or pads, diapers and the like, that are used adjacent or in contact with anatomical tissue of or in the vicinity of the external genital and/or rectal areas to thereby eliminate or reduce odor arising in or from the genital and/or rectal areas and/or odor transferred to such products from the genital and/or rectal areas.

The vaginitis treated with the methods of treatment described above encompasses inflammation, irritation, infection and diseases affecting the vagina, the vulva and/or surrounding external genital areas. The vaginitis treated with the methods of treatment described above encompasses vaginitis caused by many underlying conditions including conditions of bacterial, viral and/or fungal origin. The underlying conditions affecting the genital and/or rectal areas treated with the methods of treatment described above include bacterial vaginosis, gonorrhea, chlamydia, streptococcus, staphylococcus, trichomonas, herpes (I and II), HPV, genital warts, HIV, PID, yeast infections, thrush, candida, vaginal dryness, imbalances in vaginal pH, and odor. In women, these underlying conditions can be treated using vaginal or rectal delivery of the copper ion treatments, although vaginal delivery is preferable. In addition, vaginal delivery of the copper ion treatments can be used for contraception purposes (particularly as emergency contraception), to reduce the risk of contracting sexually transmitted diseases, and to treat odor. Many of the same underlying bacterial, viral and fungal conditions that affect the genital area in women also affect the genital area in men, and these conditions can be treated using rectal delivery of the copper ion treatments, as made possible due to the systemic therapeutic effects of the copper ion treatments. In men and women, conditions directly affecting the rectum (anus), for example hemorrhoids, and the surrounding rectal area are best treated using the methods of treatment involving rectal delivery of the copper ion treatments. The methods of treatment involving the topical application of copper ion solutions, lotions, creams, gels or foams to anatomical tissue of the external genital and/or rectal areas is particularly beneficial for treating active inflammation, irritation, infection or disease in such areas, for preventing the development of undesired body conditions and for treating odor.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A copper ion treatment for treating body conditions affecting the genital area in women, comprising
a tampon body for insertion in the vagina, said tampon body having a string attached thereto to facilitate removal of the tampon body from the vagina; and
a copper ion-containing solution carried by said tampon body, said copper ion-containing solution being composed of a biocompatible solution and copper ions contained in said biocompatible solution, said copper ions providing therapeutic effects as a result of said copper ion-containing solution contacting anatomical tissue of the vagina when said tampon body is inserted and allowed to remain therein.

2. The copper ion treatment recited in claim 1 wherein said copper ion-containing solution contains an amount of copper ions equal or substantially equal to 46 milligrams in a quantity of biocompatible solution equal or substantially equal to 7.44 ounces.

3. The copper ion treatment recited in claim 2 wherein said tampon body carries an amount of said copper ion-containing solution equal or substantially equal to 10 milliliters.

4. The copper ion treatment recited in claim 2 wherein said tampon body carries an amount of said copper ion-containing solution equal or substantially equal to 5 milliliters.

5. The copper ion treatment recited in claim 1 and further including a tampon applicator for delivering said tampon body to the vagina, said applicator comprising a hollow barrel and a hollow plunger slidable within a rearward end of said barrel, said tampon body being disposed in said barrel with said string passing through said plunger, said barrel having a forward end for introduction and advancement in the vagina, said plunger being slidable relative to and toward said barrel to eject said tampon body from said forward end of said barrel and into the vagina.

6. A copper ion treatment for treating body conditions affecting the genital or rectal areas in men and women, comprising
a suppository for insertion in the vagina or rectum, said suppository being composed of a suppository base material and a quantity of a copper ion-containing solution, said copper ion-containing solution being composed of a biocompatible solution and copper ions contained in said biocompatible solution, said copper ions being released from said suppository to contact anatomical tissue of the vagina or rectum after insertion of said suppository in the vagina or rectum, said copper ions providing therapeutic effects as a result of said copper ions contacting anatomical tissue of the vagina or rectum.

7. The copper ion treatment recited in claim 6 wherein said copper ion-containing solution contains an amount of copper ions equal or substantially equal to 46 milligrams in a quantity of biocompatible solution equal or substantially equal to 7.44 ounces.

8. The copper ion treatment recited in claim 7 wherein said suppository has a total weight equal or substantially equal to either 3 grams or 5 grams and said quantity of said copper ion-containing solution is equal or substantially equal to 5 percent, 10 percent, 20 percent or 30 percent of the total weight of said suppository.

9. The copper ion treatment recited in claim 6 and further including an applicator for delivering said suppository to the vagina or rectum, said applicator comprising a hollow barrel and a plunger slidable within a rearward end of said barrel, said barrel having a forward end for holding said suppository and for being introduced and advanced in the vagina or rectum, said plunger being slidable relative to and toward said barrel to eject said suppository from said forward end of said barrel and into the vagina or rectum

10. A method of treating vaginitis, comprising the steps of
introducing into the vagina a tampon body carrying a copper ion-containing solution;
allowing the tampon body to remain in the vagina undisturbed for 6 to 8 hours such that the copper ions from the copper ion-containing solution contact anatomical tissue of the vagina;
removing the tampon body from the vagina; and
repeating said steps of introducing, allowing and removing for a number of consecutive days.

11. The method of treating vaginitis recited in claim 10 wherein said step of introducing includes introducing a first tampon body carrying 5 milliliters of the copper ion-containing solution and further including, after said step of removing, introducing into the vagina a second tampon body carrying 5 milliliters of the copper ion-containing solution, allowing the second tampon body to remain in the vagina undisturbed for 6 to 8 hours such that the copper ions from the copper ion-containing solution contact anatomical tissue of the vagina, and removing the second tampon body from the vagina such that the first and second tampon bodies are allowed to remain in the vagina undisturbed for 6 to 8 hours within the same 24 hour period, and repeating said steps of introducing, allowing and removing for the subsequent 24 hour period and each subsequent 24 hour period thereafter until first and second tampon bodies have been allowed to remain in the vagina undisturbed for 6 to 8 hours for each of seven consecutive 24 hour periods.

12. The method of treating vaginitis recited in claim 11 wherein said step of introducing the first tampon body includes introducing the first tampon body into the vagina at bedtime, said step of removing includes removing the first tampon body in the morning, and said step of introducing the second tampon body includes introducing the second tampon body into the vagina in the morning shortly after removing the first tampon body.

13. The method of treating vaginitis recited in claim 10 wherein said step of introducing includes introducing a tampon body carrying 10 milliliters of the copper ion-containing solution and further including, after said step of removing, repeating said steps of introducing, allowing and removing for the subsequent 24 hour period and each subsequent 24 hour period thereafter such that a tampon body is allowed to remain in the vagina undisturbed for 6 to 8 hours for each of seven consecutive 24 hour periods.

14. The method of treating vaginitis recited in claim 13 wherein said step of introducing includes introducing the tampon body into the vagina at bedtime and said step of removing includes removing the tampon body from the vagina in the morning.

15. The method of treating vaginitis recited in claim 10 wherein said method is used to treat conditions including one or more of gonorrhea, chlamydia, staphylococcus, streptococcus, bacterial vaginosis, herpes (I and II), HPV, genital warts, HIV, trichomonas, PID, yeast infections, candida, thrush, vaginal dryness, imbalances in vaginal pH, odor, the risk of unwanted pregnancy and the risk of contracting STDs.

16. A method of treating vaginitis, comprising the steps of
introducing into the vagina a vaginal suppository composed of a suppository base material and a copper ion-containing solution;
allowing the vaginal suppository to remain undisturbed in the vagina for 6 to 8 hours such that copper ions from the copper ion-containing solution contact anatomical tissue of the vagina; and
repeating said steps of introducing and allowing for a number of consecutive days.

17. The method of treating vaginitis recited in claim 16 wherein said step of introducing includes introducing into the vagina a first vaginal suppository having a weight equal or substantially equal to either 3 grams or 5 grams and having either percent or 10 percent by weight of the copper ion-containing solution, and further including, after said step of allowing, introducing into the vagina a second vaginal suppository having the same weight and the same percentage of copper ion-containing solution as the first vaginal suppository, allowing the second vaginal suppository to remain in the vagina undisturbed for 6 to 8 hours such that the first and second vaginal suppositories are each allowed to remain undisturbed in the vagina for 6 to 8 hours within the same 24 hour period, and repeating said steps of introducing and allowing such that first and second vaginal suppositories are allowed to remain undisturbed in the vagina for 6 to 8 hours for each of 10 consecutive 24 hour periods.

18. The method of treating vaginitis recited in claim 17 wherein said step of introducing the first vaginal suppository includes introducing the first vaginal suppository into the vagina at bedtime and said step of introducing the second vaginal suppository includes introducing the second vaginal suppository into the vagina in the morning.

19. The method of treating vaginitis recited in claim 16 wherein said step of introducing includes introducing into the vagina a first vaginal suppository having a weight equal or substantially equal to either 3 grams or 5 grams and having 20 percent by weight of copper ion-containing solution, and further including, after said step of allowing, introducing into the vagina a second vaginal suppository having the same weight and the same percentage of copper ion-containing solution as the first vaginal suppository, allowing the second vaginal suppository to remain in the vagina undisturbed for 6 to 8 hours such that the first and second vaginal suppositories are each allowed to remain in the vagina undisturbed for 6 to 8 hours within the same 24 hour period, and repeating said steps of introducing and allowing such that first and second vaginal suppositories are allowed to remain undisturbed in the vagina for 6 to 8 hours for each of seven consecutive 24 hour periods.

20. The method of treating vaginitis recited in claim 19 wherein said step of introducing the first vaginal suppository includes introducing the first vaginal suppository at bedtime and said step of introducing the second vaginal suppository includes introducing the second vaginal suppository in the morning.

21. The method of treating vaginitis recited in claim 16 wherein said step of introducing includes introducing into the vagina a vaginal suppository having a weight equal or substantially equal to either 3 grams or 5 grams and having 30 percent by weight of the copper ion-containing solution, and further including, after said step of allowing, repeating said steps of introducing and allowing such that a vaginal suppository is allowed to remain undisturbed in the vagina for 6 to 8 hours for each of seven consecutive 24 hour periods.

22. The method of treating vaginitis recited in claim 21 wherein said step of introducing includes introducing the vaginal suppository into the vagina at bedtime.

23. The method of treating vaginitis recited in claim 16 wherein said method is used to treat conditions including one or more of gonorrhea, chlamydia, staphylococcus, streptococcus, bacterial vaginosis, herpes (I and II), HPV, genital warts, HIV, trichomonas, PID, yeast infections, candida, thrush, vaginal dryness, imbalances in vaginal pH, odor, the risk of unwanted pregnancy and the risk of contracting STDs.

24. A method of treating conditions affecting the genital or rectal areas in men and women, comprising the steps of
introducing into the rectal canal a rectal suppository composed of a suppository base material and a copper ion-containing solution;
allowing the rectal suppository to remain undisturbed in the rectal canal for 6 to 8 hours such that copper ions from the copper ion-containing solution contact anatomical tissue of the rectal canal; and
repeating said steps of introducing and allowing for a number of consecutive days.

25. The method recited in claim 24 wherein said step of introducing includes introducing into the rectal canal a first rectal suppository having a weight equal or substantially equal to either 3 grams or 5 grams and having either 5 percent or 10 percent by weight of the copper ion-containing solution, and further including, after said step of allowing, introducing into the rectal canal a second rectal suppository having the same weight and the same percentage of copper ion-containing solution as the first rectal suppository, allowing the second rectal suppository to remain undisturbed in the rectal canal for 6 to 8 hours such that the first and second rectal suppositories are each allowed to remain undisturbed in the rectal canal for 6 to 8 hours within the same 24 hour period, and repeating said steps of introducing and allowing such that first and second rectal suppositories are allowed to remain undisturbed in the rectal canal for 6 to 8 hours for each of 10 consecutive 24 hour periods.

26. The method recited in claim 25 wherein said step of introducing the first rectal suppository includes introducing the first rectal suppository at bedtime and said step of introducing the second rectal suppository includes introducing the second rectal suppository in the morning.

27. The method recited in claim 24 wherein said step of introducing includes introducing into the rectal canal a first rectal suppository having a weight equal or substantially equal to either 3 grams or 5 grams and having 20 percent by weight of the copper ion-containing solution, and further including, after said step of allowing, introducing into the rectal canal a second rectal suppository having the same weight and the same percentage of copper ion-containing solution as the first rectal suppository, allowing the second rectal suppository to remain undisturbed in the rectal canal for 6 to 8 hours, and repeating said steps of introducing and allowing such that first and second rectal suppositories are each allowed to remain undisturbed in the rectal canal for 6 to 8 hours for each of seven consecutive 24 hour periods.

28. The method recited in claim 27 wherein said step of introducing the first rectal suppository includes introducing the first rectal suppository at bedtime and the step of introducing the second rectal suppository includes introducing the second rectal suppository in the morning.

29. The method recited in claim 24 wherein said step of introducing includes introducing a rectal suppository having a weight equal or substantially equal to either grams or 5 grams and having 30 percent by weight of the copper ion-containing solution and further including, after said step of allowing, repeating said steps of introducing and allowing such that a rectal suppository is allowed to remain undisturbed in the rectal canal for 6 to 8 hours once a day for each of seven consecutive 24 hour periods.

30. The method recited in claim 29 wherein said step of introducing includes introducing the rectal suppository at bedtime.

31. The method recited in claim 24 wherein said method is used to treat conditions including one or more of gonorrhea, chlamydia, staphylococcus, streptococcus, bacterial vaginosis, herpes (I and II), HPV, genital warts, HIV, trichomonas, yeast infections, candida, thrush, hemorrhoids and odor.

32. A method of treating vaginitis comprising the steps of
introducing into the vagina a dose of copper ion treatment in the form of copper ion lotion, cream, gel or foam composed of a base material and a solution containing copper ions;
allowing the copper ion treatment to remain undisturbed in the vagina for 6 to 8 hours such that the copper ions contact anatomical tissue of the vagina; and
repeating said steps of introducing and allowing for a number of consecutive days.

33. The method of treating vaginitis recited in claim 32 wherein said step of introducing includes introducing a first dose of copper ion treatment equal or substantially equal to 5 grams and having either 5 percent or 10 percent by weight of the solution containing copper ions, and further including, after said step of allowing, introducing into the vagina a second dose of copper ion treatment the same as the first dose of copper ion treatment, allowing the second dose of copper ion treatment to remain undisturbed in the vagina for 6 to 8 hours such that the first and second doses of copper ion treatment are each allowed to remain undisturbed in the vagina for 6 to 8 hours within the same 24 hour period, and repeating said steps of introducing and allowing until first and second doses of copper ion treatment are allowed to remain undisturbed in the vagina for 6 to 8 hours for each of 10 consecutive 24 hour periods.

34. The method recited in claim 33 wherein said step of introducing the first dose of copper ion treatment includes introducing the first dose of copper ion treatment at bedtime and the step of introducing the second dose of copper ion treatment includes introducing the second dose of copper ion treatment in the morning.

35. The method recited in claim 32 wherein said step of introducing includes introducing a first dose of copper ion treatment equal or substantially equal to 5 grams and having 20 percent by weight of the solution containing copper ions, and further including, after said step of allowing, introducing into the vagina a second dose of copper ion treatment the same as the first dose of copper ion treatment, allowing the second dose of copper ion treatment to remain undisturbed in the vagina for 6 to 8 hours such that the first and second doses of copper ion treatment are each allowed to remain undisturbed in the vagina for 6 to 8 hours within the same 24 hour period, and repeating said steps of introducing and allowing until first and second doses of copper ion treatment are allowed to remain undisturbed in the vagina for 6 to 8 hours for each of seven consecutive 24 hour periods.

36. The method recited in claim 35 wherein said step of the first dose of copper ion treatment includes introducing the first dose of copper ion treatment at bedtime and the step of introducing the second dose of copper ion treatment includes introducing the second dose of copper ion treatment in the morning.

37. The method recited in claim 32 wherein said step of introducing includes introducing a dose of copper ion treatment equal or substantially equal to 5 grams and having 30 percent by weight of the solution containing copper ions, and further including repeating said steps of introducing and allowing such that a single dose of copper ion treatment is allowed to remain undisturbed in the vagina for 6 to 8 hours for each of seven consecutive 24 hour periods.

38. The method recited in claim 37 wherein said step of introducing includes introducing the dose of copper ion treatment at bedtime.

39. The method recited in claim 37 wherein said method is used to treat conditions including one or more of gonorrhea, chlamydia, staphylococcus, streptococcus, bacterial vaginosis, herpes (I and II), HPV, genital warts, HIV, trichomonas, PID, yeast infections, candida, thrush, vaginal dryness, imbalances in vaginal pH, odor, the risk of unwanted pregnancy and the risk of contracting STDs.

40. A method of treating conditions affecting the genital or rectal areas in men and women, comprising the steps of
topically applying a copper ion treatment in the form of copper ion lotion, cream, gel, foam or solution to anatomical tissue of the external genital or rectal areas; and
repeating the step of topically applying as needed to treat active inflammatory conditions and to prevent the development of undesired conditions.

41. A method of female contraception, comprising the steps of
introducing into the vagina, within 4 to 6 hours or less following sexual intercourse, a copper ion treatment composed of a copper ion-containing solution and a carrier for the copper ion-containing solution;
allowing the copper ion treatment to remain undisturbed in the vagina for 6 to 8 hours; and
continuing to introduce into the vagina additional copper ion treatments and allowing them to remain undisturbed in the vagina for 6 to 8 hours such that at least one copper ion treatment is introduced into the vagina each day for a number of consecutive days.

42. The method recited in claim 41 wherein the carrier is a tampon body supplied with the copper ion-containing solution.

43. The method recited in claim 41 wherein the carrier is a lotion base material, a cream base material, a gel base material or a foam base material.

44. The method recited in claim 41 wherein the carrier is a vaginal suppository.

45. The method recited in claim 41 wherein two of the copper ion treatments are introduced into the vagina each day for 7 consecutive days.
